(19) United States
(12) Patent Application Publication
(10) Pub. No.: US 2004/0009174 A1
(43) Pub. Date: Jan. 15, 2004

(54) Method of treating asthma

(76) Inventors: Gregory Martin Arndt, Malabar (AU); Judith Lee Black, Sydney (AU); Janette Kay Burgess, Kingsford (AU); Nicholas Henry Hunt, Newtown (AU); Robert Andrew Pack, Pyrmont (AU)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/321,145

(22) Filed: Dec. 17, 2002

Related U.S. Application Data
(60) Provisional application No. 60/341,453, filed on Dec. 18, 2001.

(51) Int. Cl. A61K 38/17; A61K 39/395

(52) U.S. Cl. 424/146.1; 514/2

(57) ABSTRACT

The present invention relates to methods for inhibiting the binding of OX40 and OX40L in smooth muscle cells to reduce inflammatory responses in smooth muscle containing tissue.

### FIELD OF THE INVENTION

The present invention claims priority from U.S. Provisonal Patent Application No. 60/341,453 filed Dec. 18, 2001 and entitled “Methods of Treating Asthma” the contents of which is hereby incorporated by reference in its entirety.

The present invention relates to a method for treating an inflammatory disease of smooth muscle tissue. In particular embodiments, the smooth muscle tissue is airway smooth muscle. In a particular application of the embodiments of this invention, the method may be used for the treatment of asthma. In other particular embodiments, the smooth muscle tissue is vascular smooth muscle or gut smooth muscle.

### BACKGROUND OF THE INVENTION

While it has been recognised that increased airway smooth muscle (ASM) cell proliferation and airway hyperresponsiveness are key features of persistent asthma, the mechanisms that underlie these processes have not been well characterised to date. It is, however, known that ASM cells interact with and respond to cells, soluble mediators and cytokines in their immediate vicinity to produce further cytokines and proteins that have the potential to influence airway inflammation and remodelling. For example, activated T cells adhere to human cultured airway smooth muscle cells (HASMs) via integrins and the cell adhesion molecule, CD44, to induce HASM DNA synthesis in a contact-dependent manner (Lazaar et al., 1994). Cytokines released from activated T cells have been observed to up-regulate the expression of Class II major histocompatibility antigens (MHC II) and the intercellular adhesion molecule, ICAM-1 (CD54), on the surface of HASMs (Lazaar et al., 1997), although the HASMs were unable to present antigen to CD4⁺ T cells. Further, CD40 has been reported to be present at a low constitutive level on HASMs (Lazaar et al., 1998) and is up-regulated by TNF-α or IFN-γ. The stimulation of the HASMs with trimerised human CD40 ligand (CD40L) leads to increased interleukin-6 (IL-6) secretion, increases in cytosolic calcium and activation of NF-κB (Lazaar et al., 1998). Thus, CD40 signalling has an important role in the development of an immune response. Other molecules are also implicated in inflammatory responses including CD86, the CD28 ligand, which is constitutively expressed on mature dendritic cells and is able to provide all antigen specific CD4⁺ T cells with an initial CD28 signal after the interaction of the antigen/MHC II complex with the T cell receptor (TCR) (Toeliner et al., 1998). Immediately after TCR stimulation, the CD40 ligand (CD40L) is expressed on the surface of the CD4⁺ T cells enabling these cells, in turn, to activate the antigen presenting cells (APCs)/B cells through CD40 which is constitutively expressed on the T cell surface (Foy et al., 1996; Grewal and Flavell, 1998). It has further been found that the induction of B7-1 (CD80) and increased or sustained expression of CD44H, ICAM-1 and B7-2 (CD86), which are required to provide the co-stimulatory signals to the T cells, are dependent on the interaction between CD40 and CD40L (Van Gool et al., 1996; Akiba et al., 1999; Weinberg et al., 1999; Evans et al., 2000). Moreover, CD40 stimulation of B cells results in the up-regulation of nuclear factor (NF)-κB (Lalmanach-Girard et al., 1993; Berberich et al., 1994), as well as the up regulation of a number of other transcription factors including nuclear factor of activated T cells (NFAT) (Francis et al., 1995) and activator protein I (AP-1) (Francis et al., 1995; Huo and Rothstein, 1995).

Activated APCs express OX40 ligand (OX40L), a member of the TNF receptor family, on their surfaces within two days of stimulation through CD40 and this expression is sustained for at least seven days (Murata et al., 2000). Naive T cells express OX40, the receptor for OX40L, on their surfaces following the initial CD28 signal. This has been demonstrated both in vitro and in vivo (Walker et al., 1999) and has been reported to peak around 48 hr after TCR stimulation (Gramaglia et al., 1998), so that the expression of OX40 on the CD4⁺ T cells and OX40L on the APCs coincides. The sequential expression of CD40-CD40L and OX40-OX40L suggests that the interaction of OX40 with its ligand is involved in the later phase of T cell priming rather than the initial signals.

Interactions between OX40 and OX40L have been shown to result in increased proliferation of T cells expressing OX40 (Baum et al., 1994; Godfrey et al., 1994). Enhanced proliferation and differentiation of B cells expressing OX40L also have been demonstrated (Calderhead et al., 1993; Stuber et al., 1995). Further, several groups have reported the functional consequences of the bi-directional signalling between OX40 and OX40L, with CD4+0×40⁺ T cells producing IL-4 (Flynn et al., 1998) and OX40L⁺ dendritic cells producing TNF-α, IL-12, IL-1β and IL-6 (Ohshima et al., 1997) as a result of the signalling cascade triggered when OX40 and OX40L interact.

The interaction between OX40-OX40L has been implicated in the pathogenesis of several inflammatory diseases. Blocking of OX40-OX40L interactions in inflammatory bowel disease reduced the disease symptoms (Higgins et al., 1999). In addition, the in vivo administration of soluble OX40 to mice at the onset of actively induced or adoptively transferred experimental allergic encephalomyelitis (EAE) reduced ongoing signs of the disease and the animals recovered more rapidly (Weinberg et al., 1999). These researchers concluded that the OX40L expressed on the central nervous system (CNS) APCs provides an important co-stimulatory signal to EAE effector T cells. OX40 has been identified on T cells isolated from inflammatory sites in several disease states including EAE (Buenafe et al., 1996; Weinberg et al., 1996a), rheumatoid arthritis (Weinberg et al., 1996b), graft versus host disease (Tittle et al., 1997) and also on lymphocytes infiltrating into tumors (Vetto et al., 1997).

The events following CD40-CD40L interaction could play an important role in signalling between cells present in the airways. To date, there are no reports of the expression of OX40 or OX40L on smooth muscle cells. In examining the expression of OX40L mRNA and protein on human ASM cells the inventors have analyzed the functional consequences of ligand engagement with OX40 by measuring cytokine secretion and downstream signalling events. These studies showed that ASM express OX40L and that, when engaged, this cell surface molecule stimulates the expression of the pro-inflammatory cytokine IL-6 and activates PKCβ2, each of which in turn can influence the pathology of the airway. More specifically, the studies showed that upon interaction of the OX40L on HASM cells with soluble OX40, PKCβ2 migrates to the cellular membrane and the production of the pro-inflammatory cytokine IL-6 is induced. In addition, the pro-inflammatory cytokine TNFα is shown to induce both CD40 and OX40L in HASM cells, implicating the activation of OX40L in an inflammatory cascade involving ASM in human asthma. Thus, it has been recognised by the present inventors that the OX40L surface protein represents a potential target for treatment of persistent asthma, as well as other inflammatory diseases.

### SUMMARY OF THE INVENTION

Thus, in a first aspect, the present invention provides a method of treating and/or preventing an inflammatory disease or condition of smooth muscle tissue in a subject, said method comprising administering to said subject an effective amount of an agent which inhibits or blocks interaction between OX40 and OX40L.

In a second aspect, the present invention provides a method of inhibiting or preventing induction of IL6 production in inflammatory diseases or conditions of smooth muscle tissue in a subject, said method comprising administering to said subject an effective amount of an agent which inhibits or blocks interaction between OX40 and OX40L.

In a third aspect, the present invention provides a method of inhibiting or preventing translocation of PKCβ2 to cellular membranes in inflammatory diseases or conditions of smooth muscle tissue in a subject, said method comprising administering to said subject an effective amount of an agent which inhibits or blocks interaction between OX40 and OX40L.

In a fourth aspect, the present invention provides a method of inhibiting or preventing T cell priming in inflammatory diseases or conditions of smooth muscle tissue in a subject, said method comprising administering to said subject an effective amount of an agent which inhibits or blocks interaction between OX40 and OX40L.

In a fifth aspect, the present invention provides the use of an agent which inhibits or blocks interaction between OX40 and OX40L for the preparation of a medicament for treating and/or preventing an inflammatory disease or condition of smooth muscle tissue.

In a sixth aspect, the present invention provides the use of an agent which inhibits or prevents induction of IL6 production for the preparation of a medicament for treating and/or preventing an inflammatory disease or condition of smooth muscle tissue in a subject.

In a seventh aspect, the present invention provides the use of an agent which inhibits or prevents translocation of PKCβ2 to cellular membranes for the preparation of a medicament for treating and/or preventing an inflammatory disease or condition of smooth muscle tissue in a subject.

In an eighth aspect, the present invention provides the use of an agent which inhibits or prevents T cell priming for the preparation of a medicament for treating and/or preventing an inflammatory disease or condition of smooth muscle tissue in a subject.

In a ninth aspect, the present invention provides an agent which inhibits or blocks interaction between OX40 and OX40L when used in the treatment and/or prevention of an inflammatory disease or condition of smooth muscle tissue in a subject.

In a tenth aspect, the present invention provides an agent which inhibits or prevents induction of IL6 production when used in the treatment and/or prevention of an inflammatory disease or condition of smooth muscle tissue in a subject.

In an eleventh aspect, the present invention provides an agent which inhibits or prevents translocation of PKCβ2 to cellular membranes when used in the treatment and/or prevention of an inflammatory disease or condition of smooth muscle tissue in a subject.

In a twelfth aspect, the present invention provides an agent which inhibits or prevents T cell priming when used in the treatment and/or prevention of an inflammatory disease or condition of smooth muscle tissue in a subject.

In a thirteenth aspect, the present invention provides a method of screening a molecule for an ability to inhibit or block interaction between OX40 and OX40L in smooth muscle cells, said method comprising the steps of:

(a) contacting a molecule with smooth muscle cells obtained from a subject;

(b) treating the cells with labelled OX40;

(c) washing the treated cells to remove any unbound labelled OX40; and

(d) detecting the level of labelled OX40.

In a preferred embodiment, the detecting step further compares the amount of label detected with the amount of label detected in cell samples receiving no molecule or using test molecules that do not bind to the OX40L. Control levels of labelled OX40 for the method of screening of the thirteenth aspect may be obtained in a number of ways all of which would be general knowledge for the skilled artisan. In some preferred embodiments, for example, the control level of labelled OX40 can be obtained by the following steps:

(a) obtaining a collection of smooth muscle cells from a subject;

(b) treating the collection from (a) with labelled OX40;

(c) washing the treated collection from (b) to remove any unbound labelled OX40; and

(d) assessing the washed collection from (c) to obtain a control level of labelled OX40.

For the purpose of the method of screening of the thirteenth aspect, any of a number of labels may be used in labelling OX40. In some preferred embodiments, fluorescent labelling is used. In another embodiment radiolabelled OX40 is used.

The invention further relates to a composition comprising OX40 protein or a fragment thereof in combination with isolated smooth muscle cell membranes comprising OX40L. The cells are preferably obtained from airway passage tissue, colon tissue or aorta. In one embodiment, the smooth muscle cell membranes are obtained from a cell lysate of smooth muscle cells and in another embodiment the isolated smooth muscle cell membranes are part of intact isolated smooth muscle cells.

The invention also relates to a fusion protein of OX40 with the immunoglobulin Fe portion. Preferably the fusion protein comprises at least a portion of the human OX40 sequence in combination with the Fe portion of human IgG. In one aspect the fusion protein comprises amino acids 1-220 of OX40. Those of ordinary skill in the art will appreciate that the size of the OX40 fragment can be increased or decreased as long as the fragment includes that portion of OX40 that binds to OX40L.

In another embodiment, the invention relates to a method of reducing inflammation in smooth muscle tissue in a subject, the method comprising administering to said subject an effective amount of an agent which inhibits OX40L binding to OX40. The invention also relates to a method of reducing the induction of IL6 production in smooth muscle tissue in a subject, said method comprising administering to said subject an effective amount of an agent which inhibits OX40L binding to OX40. Additionally the invention relates to a method of inhibiting translocation of PKCβ2 to cellular membranes in smooth muscle cells comprising administering to said subject an effective amount of an agent which inhibits or blocks interaction between OX40 and OX40L.

In one preferred embodiment, the invention relates to a method for inhibiting translocation of PKCβ2 to the cellular membranes of smooth muscle cells comprising OX40L, the method comprising the step of inhibiting the ability of OX40 to bind to OX40L. Preferably the smooth muscle cell is obtained from airway tissue, colon tissue or aorta tissue. In one aspect of this embodiment, the cellular membranes of a smooth muscle cell are part of intact smooth muscle cells and the smooth muscle cells can be isolated cells or part of an isolated tissue or tissue sample. Alternatively the cellular membranes of smooth muscle cells are part of a cell lysate. Preferably the inhibiting step comprises contacting the cell membranes with an agent capable of inhibiting OX40 binding to OX40L. In one embodiment of this method the agent binds to OX40 and in another embodiment the agent binds to OX40L. In another embodiment, the agent is an antibody or antibody fragment capable of binding to OX40 or alternatively is an agent capable of binding to OX40L. In yet another embodiment the inhibiting step comprises inhibiting expression of OX40L and wherein the cellular membranes of smooth muscle cells comprise intact smooth muscle cells. In one aspect of this embodiment the inhibiting step further comprises introducing a gene expression reduction agent into the cell. Preferably the gene expression reduction agent is selected from the group consisting of DNAzymes, ribozymes, antisense RNA, dominant negative polypeptides, genetic suppression elements or intracellular antibodies.

This invention also relates to an antibody or antibody fragment capable of inhibiting OX40 binding to OX40L on smooth muscle cells.

The invention further relates to a method of identifying an agent that is capable of inhibiting binding of OX40 and OX40L in a smooth muscle cell preparation, said method comprising the steps of: (a) combing the agent with isolated smooth muscle cell membranes; (b) adding OX40; and (c) detecting binding of OX 40 to OX40L. In one aspect of this method a reduction in the binding of OX40 to OX40L identifies a candidate agent. It is contemplated that either OX 40, OX40L or both can be labelled to promote detection. The smooth muscle cell membranes can be part of intact smooth muscle cells, part of a tissue sample or part of a cell lysate.

### BRIEF DESCRIPTION OF THE ACCOMPANYING FIGURES

FIG. 1. Shows the expression of OX40L mRNA in human airway smooth muscle cells: OX40L mRNA expression measured by real time RT-PCR in non-asthmatic and asthmatic HASM cells. Ct values are expressed after normalisation for rRNA expression level within each sample (n=6).

FIG. 2. Shows the expression of OX40L mRNA in human airway smooth muscle captured from tissue biopsies: OX40L mRNA expression measured by real time RT-PCR. The histograms indicate the cycle number required to cross the Ct threshold. The numbers 2574 and 2862 represent independent patients. Samples “2826+RNALater” and “2826 (Organ)+RNALater” represent the results from tissues deposited directly into RNALater solution and tissues recovered from organ bath prior to treatment with RNALater, respectively.

FIG. 3. Expression of OX40L protein on human airway smooth muscle cells using ELISA and immunoprecipitation: (A) OX40L cell surface expression measured by ELISA in non-asthmatic □ and asthmatic □ cells (n=4, *Absorbance significantly greater than mIgG₁, P<0.05); and (B) Cell surface expression of OX40L in the above cells as detected by flow cytometry

FIG. 4. Detection of OX40L protein surface expression on human airway smooth muscle cells using flow cytometry: (A) Both the 5A8 monoclonal antibody and OX40:Fc construct detect OX40L protein expression in human embryonic kidney cells transfected with an OX40L expression plasmid. The top row of plots represent the results obtained using the 5A8 antibody while the bottom row shows the results using the OX40:Fc detection system. Human 293 cells containing the vector control are indicated by “Vector” and the cells expressing OX40L (and GFP) are labelled “OX40L”. The controls for 5A8 and OX40:Fc are IgG1 and Fc, respectively. The Y-axis in each plot represents OX40L signal and the X-axis shows GFP signal; and (B) Detection of OX40L protein expression on HASM cells from non-asthmatic, non-asthmatic/sensitised and asthmatic patient samples. The OX40L phenotypic profiles for each patient class are displayed in three different groups (high, medium and low) according to the criteria indicated in Table 1. Solid peak represents isotype control and open peak indicates binding of OX40:Fc to OX40L.

FIG. 5. PKCβ2 response to incubation with OX40 receptor: Graph shows means±SE from 3 separate experiments (for the 10 minute time point n=2, and for the 24 h time point n=1) (*P<0.05)).

FIG. 6. OX40L enhances IL-6 release by HASM cells upon treatment with soluble OX40:Fc: (A) IL-6 released by HASM cells exposed to 1% FCS, human IgG Fc fragment or OX40:Fc at day 4 (D4) and day 10 (D10) (D4 n=2, D10 n=6*P<0.05); and (B) Cell surface expression of OX40L in the above cells as detected by flow cytometry. The peak indicated by the grey line represents isotype control and the peak indicated by the black line shows binding of OX40:Fc.

FIG. 7. Additive effect of OX40L and CD40 ligation on IL-6 release in HASM cells. HASM cells were seeded at 1×10⁴ cells/cm² in 96 well plates and grown to sub-confluence (Day 4) or confluence (Day 10). The cells were then quiesced for 24 h with 1% FCS after which the OX40:Fc construct, an Fc control construct, recombinant human soluble CD40L (CD40L), and OX40:Fc in combination with CD40L were added to the cells at saturating concentrations (1 μg/ml). Supernatants were collected 24 and 48 h after addition of the constructs/cytokines to assay expression of IL-6 by ELISA.

FIG. 8. Pro-inflammatory cytokine TNFα induces CD40 and OX40L surface expression on HASM cells: (A and B) Induction of OX40L surface expression on subconfluent and confluent HASM cells following 24 h treatment with TNFα; and (C and D) Time course of induction in the surface expression of CD40 and OX40L on HASM cells treated with TNFα.

FIG. 9. The OX40L-specific monoclonal antibody 5A8 blocks OX40-OX40L interaction on the HASM cells: (A) OX40L-specific monoclonal antibody 5A8 blocks binding of soluble OX40:Fc in HASM cells transiently transfected with a plasmid expressing OX40L cDNA; and (B) OX40L-specific monoclonal antibody 5A8 blocks binding of soluble OX40:Fc in HASM cells expressing endogenous OX40L. To assay the ability of the 5A8 monoclonal antibody to block the interaction of the OX40:Fc construct and OX40L on HASM cells expressing high levels of OX40L, HASM cells were harvested from flasks and the cell suspensions were incubated with high concentrations of 5A8 (10-100 μg/ml) for 10 min at room temperature prior to detection of OX40L expression using the OX40:Fc detection system.

FIG. 10. Expression of OX40L on smooth muscle cells derived from human colon and aorta. Primary human smooth muscle cells from colon or aorta were seeded at 1×10⁴ cells/cm² and grown for 48 h. The cells were then quiesced for 24 h with 1% FCS after which TNF-α was added at 10 mg/ml for 48 h. (A) Expression of OX40L and CD40 on the surface of human colon smooth muscle cells and induction of these surface proteins by TNF-α; and (B) Expression of OX40L and CD40 on the surface of human aortic smooth muscle cells and induction of these surface proteins by TNF-α.

FIG. 11. Time course of IL-6 release by ASM cells following OX40L stimulation. IL-6 released by asthmatic and non-asthmatic ASM cells exposed to human IgG Fe fragment (and respectively) or OX40:Fc (and respectively) over time, asthmatic n=3, non-asthmatic n=3. Data is expressed as a percentage of IL-6 released in 1% FBS alone at the relevant time point. Graph shows mean±SE. *Significant increase in IL-6 release compared to time 0 P<0.01; # Significant difference in overall OX40:Fc response compared to human IgG Fe fragment response P<0.01; §Significant difference in asthmatic response compared to non-asthmatic response P<0.02.

### DETAILED DISCLOSURE OF THE INVENTION

In some preferred embodiments, the present invention provides a method, and use of an agent in the preparation of a medicament, for treating an inflammatory disease or condition of smooth muscle tissue. Preferably, the inflammatory disease or condition to be treated is an inflammatory disease or condition of smooth muscle tissue of the airways and, particularly, asthma. While not wishing to be bound by theory, it is believed that OX40L on the surface of HASM cells has an integral role in the inflammatory reaction associated with asthma by interacting with and stimulating T cells expressing the OX40 receptor and, possibly, by also mediating reverse signalling into ASM cells, both of which involve production of the pro-inflammatory cytokine IL-6 and translocation of PKCβ2 to the cellular membrane which has been implicated as having a function in cell turnover. Thus, by inhibiting or blocking the interaction of OX40 receptor with OX40L, the inflammatory reaction associated with asthma will be substantially reduced and preferably prevented.

In addition, the present inventors consider that in the light of their finding that OX40L is expressed by ASM, it is likely that OX40L is also expressed by vascular and gut smooth muscle tissue where it may respectively play a role in cardiovascular disease and inflammatory gut diseases. Thus, the present invention also extends to a method, and use of an agent in the preparation of a medicament, for treating cardiovascular disease or an inflammatory gut disease or condition.

The agent for inhibiting or blocking the interaction of OX40 and OX40L preferably blocks binding of OX40 to OX40L expressed on the surface of the smooth muscle cells. Described hereinafter is a monoclonal antibody, 5A8, which specifically binds to OX40L and demonstrates that inhibitors of the interaction of OX40 and OX40L results in a reduction in the inflammatory response in smooth muscle. As will be readily understood to those of ordinary skill in the art of receptor/ligand interactions, monoclonal antibodies that interrupt the binding of OX40 and OX40L are just one form of an agent suitable for use in the present invention. Other antibodies can be used that specifically bind to OX40, particularly those that bind to an OX40 antigenic determinant located at the cell surface of a cell expressing OX40 such as a T cell. Fragments of anti-OX40L and anti-OX40 antibodies such as Fab and F(ab′)₂ fragments, as well as recombinant antibodies or fragments thereof (e.g. scFv) directed against OX40 or OX40L are also suitable. Further suitable agents include peptide and polypeptide fragments of OX40 and OX40L which include at least part of the site(s) involved in the interaction between OX40 and OX40L (e.g. peptide and polypeptide fragments which mimic a binding site on the OX40 or OX40L) and can thereby inhibit or block the interaction between OX40 and OX40L.

Peptide mimetics of such fragments are also contemplated. Such peptide mimetics may be designed using any of the methods well known in the art for designing mimetics of peptides based upon peptide sequences in the absence of secondary and tertiary structural information (see Kirshenbaum et al., 1999). For example, peptide mimetics may be produced by modifying amino acid side chains to increase the hydrophobicity of defined regions of the peptide (e.g. substituting hydrogens with methyl groups on aromatic residues of the peptides), substituting amino acid side chains with non-amino acid side chains (e.g. substituting aromatic residues of the peptides with other aryl groups), and substituting amino- and/or carboxy-termini with various substituents (e.g. substituting aliphatic groups to increase hydrophobicity). Alternatively, suitable peptide mimetics may be so-called peptoids (i.e. non-peptides) which include modification of the peptide backbone (i.e. introducing amide bond surrogates by, for example, replacing the nitrogen atoms in the backbone with carbon atoms), or include N-substituted glycine residues, one or more D-amino acids (in place of L-amino acid(s)) and/or one or more α-amino acids (in place of β-amino acids or γ-amino acids). Further peptide mimetic alternatives include “retro-inverso peptides” where the peptide bonds are reversed and D-amino acids assembled in reverse order to the order of the L-amino acids in the peptide sequence upon which they are based, and other non-peptide frameworks such as steroids, saccharides, benzazepine 1,3,4-trisubstituted pyrrolidinone, pyridones and pyridopyrazines.

Other suitable agents for use in the present invention include gene expression reduction agents designed to prevent or reduce expression of OX40 and/or OX40L such as genetic suppression elements, ribozymes, RNAi or antisense RNA targeted against OX40 or OX40L mRNA (or DNA which when introduced into cells of the target smooth muscle tissue cause expression of the ribozymes or antisense RNA, e.g. viral vectors including sequences encoding the genetic suppression elements, ribozymes, RNAi or antisense RNA), DNAzymes, and dominant negative polypeptides and intracellular or catalytic antibodies (or DNA which when introduced into cells of the target smooth muscle tissue cause expression of the dominant negative polypeptides and intracellular or catalytic antibodies).

In addition, agents suitable for use in the present invention may be identified by screening suitable combinatorial compound libraries for compounds which bind to one or other of OX40 and OX40L or which otherwise inhibit or block the interaction between OX40 and OX40L, virtual screening/database searching (Bissantz et al., 2000) and rational drug design techniques well known in the art (Houghten et al., 2000).

In the method of the present invention, the agent may be administered to the subject (which is preferably human, but which may be selected from other animals) by any route that successfully delivers the agent to the target smooth muscle tissue. Preferred methods of administering include administration of the agent by oral, buccal or nasal routes (e.g. with oral dosage forms and nasal sprays), and intravenous, intramuscular or intraperitoneal administration.

Accordingly, the agent may be formulated as a medicament in the form of, for example, a syrup, nasal spray, a tablet, a capsule, a caplet, or liquid solution or suspension. The agent may therefore be administered in combination with a variety of liquid or solid pharmaceutically-acceptable carriers and excipients including inert diluents (e.g. lactose, calcium carbonate, calcium phosphate and sodium phosphate), binding agents (e.g. starch and gelatin), lubricants (e.g. magnesium stearate, stearic acid or talc), and granulating and disintegrating agents (e.g. corn starch and alginic acid).

An “effective amount” of an agent will be an amount of the agent which is effective to achieve a desired therapeutic response (e.g. a desired prevention or reduction in an inflammatory reaction) and will depend on a number of factors including the identity of the particular agent, the nature of the system to which it is to be administered or applied, and the type and severity of the inflammatory disease or condition to be treated.

The terms “comprise”, “comprises” and “comprising” as used throughout the specification are intended to refer to the inclusion of a stated step, component or feature or group of steps, components or features with or without the inclusion of a further step, component or feature or group of steps, components or features.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

This invention will be better understood by reference to the Examples that follow, but those skilled in the art will readily appreciate that these are only illustrative of the invention as described more fully in the claims which follow thereafter. Additionally, throughout this application, various publications are cited. The disclosure of these publications is hereby incorporated by reference into this application to describe more fully the state of the art to which this invention pertains.

### EXAMPLES

Materials and Methods:

The following materials and methods were used in the Examples 1 to 8 hereinafter.

Chemicals

The following compounds were obtained from the sources given in parentheses: Dulbecco's modified Eagle's medium (DMEM), Dulbecco's phosphate buffered saline (PBS), penicillin, streptomycin, amphotericin B, trypan blue, (Life Technologies, Heidelberg, Australia); ethylenediaminetetra-acetic acid disodium salt, (Ajax, Australia); foetal calf serum (FCS) (Commonwealth Serum Laboratories, Melbourne, Australia); bovine serum albumin (BSA), TNFα, phytohaemagglutinin (PHA), and phorbol myristate acetate (PMA) (Sigma, St Louis, Mich.).

Antibodies and Recombinant Proteins

Fluorescein (FITC)-conjugated monoclonal anti-α smooth muscle actin (mouse IgG₂ₐ isotype), monoclonal anti-calponin (mouse IgG₁), FITC-conjugated goat anti-mouse IgG (Sigma, St Louis, Mich.), FITC-conjugated AffiniPure goat anti-mouse IgG (Jackson ImmunoResearch Laboratories, Inc., West Grove, Pa.) R-phycoerythrin (R-PE)-labelled polyclonal anti-mouse Ig antibody (Becton Dickinson, San Jose, Calif.), PE-labelled F(ab′)₂ goat anti-human IgG F(c) (Rockland, Gilbertsville, Pa.) and streptavidin-HRP (Amersham, United Kingdom) were purchased as indicated.

All monoclonal antibodies were of the IgG class. MOPC21 (murine IgG₁ control), IgG₂ₐ isotype control, monoclonal rat anti-human IL-6 and biotinylated monoclonal rat anti-human IL-6 were purchased from PharMingen (San Jose, Calif.). Mouse anti-human CD40 and CD40L monoclonal antibodies were obtained from Immunotech (Marseille, France) and mouse anti-human HLA-DP, DQ, DR monoclonal antibodies and anti-human HLA Class I monoclonal antibody from DAKO (Glostrup, Denmark). Monoclonal anti-human CD134 (±PE) was from Ancell Corporation (Bayport, Minn.). 5A8, a monoclonal mouse anti-human gp34 (OX40 ligand) antibody, was a generous gift from Professor Yuetsu Tanaka (Okinawa, Japan). Rabbit anti-human anti-PKC antibodies were purchased from Santa Cruz Biotechnology, while the goat anti-rabbit secondary (HRP-conjugated) antibody was from Sigma (St Louis, Mich.). The anti-human CD54 (ICAM-1) and anti-human CD106 (VCAM-1) monoclonal antibodies were purchased from Pharmingen (San Jose, Calif.).

Soluble human recombinant OX40 [CD134]:Fc and recombinant human soluble CD40L were purchased from Alexis Corporation (San Diego, Calif.). The human IgG Fc control fragment was purchased from Jackson ImmunoResearch Laboratories, Inc. (West Grove, Pa.).

Cell Culture

Human ASM cells were obtained from non-asthmatics and asthmatics by methods adapted from those previously described (Johnson et al., 1995; Hawker et al., 1998; Carlin et al., 1999). Non-asthmatic human ASM was obtained from bronchial airways of patients undergoing resection for either lung transplantation or carcinoma. Asthmatic ASM was obtained from: patients undergoing resection for lung transplantation; 1 patient dying in status asthmaticus; and patients undergoing deep endobronchial biopsies obtained by flexible bronchoscopy. Pure ASM bundles were dissected free from surrounding tissue using a dissecting microscope. The small pieces of muscle bundles were then plated in 12 cm² flasks as previously described (Johnson et al., 1995; Hawker et al., 1998; Carlin et al., 1999). ASM cell characteristics were determined by immunofluorescence and light microscopy. Cells were stained with antibodies against α-smooth muscle actin and calponin while omission of the primary antibody was used as a control (Durand-Arczynska et al., 1993).

Real Time Reverse Transcription Polymerase Chain Reaction (RT-PCR)

Total RNA was extracted from the cells using the RNeasy Mini Kit (Qiagen, Clifton Hill, Australia) according to the manufacturer's instructions, and the RNA concentration determined spectrophotometrically. After extraction, samples were stored at −80° C. until use. OX40L forward primer (5′-TCACCTACATCTGCCTGCACTT-3′ SEQ ID NO: 1), reverse primer (5′-GAAACCTTTCTCCTTCTTATATTCGGTA-3′ SEQ ID NO:2) and internal probe (FAM-5′-TGCTCTTCAGGTATCACATCGGTATCCTCG-3′-TAMRA, SEQ ID NO:3) were designed using Primer Express (ABI Prism) and synthesized by PE Applied Biosystems (Foster City, Calif.). The primers were selected to span two adjacent exons of the gene to avoid amplification of genomic DNA. The human OX40L cDNA sequence is available from GenBank as NM_{—}003326 and the protein is available as NP_{—}003317. The mouse OX40L cDNA sequence is available from GenBank as U12763 and the protein sequence is available from GenBank as AAA21871. The human OX40 receptor cDNA is available from GenBank as NM_{—}003327 and the protein is available as NP_{—}003318.

Real-time RT-PCR was prepared using the TaqMan® One-Step RT-PCR Master Mix Reagents Kit (PE Applied Biosystems). For precise quantitative analysis of gene expression, the Pre-developed TaqMan® Assay Reagents [Endogenous Control. Ribosomal RNA Control (18s rRNA)] (PE Applied Biosystems) was included in the RT-PCR reactions. One hundred nanogram of total RNA was analyzed in a 25 μl reaction containing 1× Master Mix, 1× MultiScribe and RNase Inhibitor Mix, 300 nM OX40L forward primer, 300 nM OX40L reverse primer, 100 nM OX40L probe, 1×18s rRNA Primer and Probe Mix. RT-PCR reaction was performed in the ABI Prism 7700 Sequence Detection System (PE Applied Biosystems). The thermal cycle conditions consisted of reverse transcription at 48° C. for 30 minutes, denaturation at 95° C. for 10 minutes, followed by 40 cycles of 95° C. for 15 seconds and 60° C. for 1 minute. Data from the reaction were collected and analyzed by the complementary computer software.

Plasmid Construction and Cell Transfection

The full-length cDNA of OX40L was amplified by PCR using pSGP34-1 (Miura et al., 1991) as a template and the forward primer (5′ GCGCTCTAGAAAGATCCCTCGAGATCCA 3′ SEQ ID NO:4) and reverse primer (5′ ATACAGTAACTTTTGCCTAGATCTCGCG 3′ SEQ ID NO:5). The PCR primers used included Xbal restriction enzyme sites enabling the OX40L cDNA to be cloned as an XbaI fragment under the control of the CMV promoter in an episomal expression plasmid. This vector (designated pCMG-OX40L(s)) contained a separate expression cassette for the GFP marker.

HASM cells and human embryonic kidney 293 cells were transfected with pCMG-OX40L(s) by electroporation. A ratio of 10 μg plasmid DNA per 2.5×10⁶ cells was routinely used to electroporate cells in 0.5 ml PBS at 350 and 250 V for the HASM and 293 cells, respectively. Cells were harvested for flow cytometry routinely at 24, 48 and/or 72 h after electroporation.

Flow Cytometry

Adherent HASM and 293 cells were harvested from flasks for flow cytometry analysis using 0.05% trypsin and 0.53 mM EDTA and washed in 0.2% (w/v) BSA in PBS. Cell suspensions were stained for expression of cell surface proteins using appropriate concentrations of mouse anti-human antibodies for 10 min at RT followed by two washes. The cells were then incubated with PE-labelled anti-mouse Ig secondary antibody for 10 min at RT followed again by two washes. Flow cytometry was then performed on a FACSort (Becton Dickinson) using Cell Quest software (Becton Dickinson). The expression of OX40L was detected by either 5A8, a mouse anti-human monoclonal antibody, as above or a OX40:Fc construct (a cysteine-rich region of human OX40 receptor fused to the Fc portion of human IgG) followed by a PE-labelled goat anti human Fc (IgG) detection antibody. Since it was possible that the OX40:Fc construct could be binding to Fcγ receptors on the 293 cells by the Fc portion of the construct, a control Fc fragment of human IgG was also tested but found to be negative for binding to the cells. ELISA

Cells were analyzed for surface expression of the OX40L using the ELISA method as described by Hicks and colleagues (Hicks et al., 1994) with slight modifications. Briefly, non-asthmatic and asthmatic ASM cells were grown for 7 days in 96 well plates in 10% FBS, DMEM. The supernatant was removed and the cells dried overnight at room temperature. The plate was stored at −20° C., in the presence of desiccant, until analysis. After defrosting the plate was washed twice with 0.05% (v/v) Tween-20/PBS and blocked with 2% (w/v) BSA/PBS for 1 h at room temperature. The wells were washed and monoclonal antibodies, final concentration of 2 μg/ml (except 5A8 final concentration of 10 μg/ml), added before overnight incubation at 4° C. The plate was washed five times before the goat anti-mouse HRP-conjugated secondary antibody in 1% BSA/PBS/0.05% Tween-20 was added and incubated at room temperature for 1 h. The plate was washed seven times and the TMB substrate added according to the manufacturer's instructions. Colour development was stopped by the addition of phosphoric acid and the optical density read at 450 nm.

For measurement of release of cytokines into the cell supernatant, cells were grown in 6-well plates for 7 days to confluence in 10% FCS, DMEM then quiesced for 24 h in 1% FCS. OX40:Fc or human Fc was added to cells at 1 μg/ml in 1% FCS for 24 h, and the supernatants collected for analysis. The quantities of the following cytokines were tested using commercial ELISA kits according to the manufacturers' instructions: IL-10, IL-4, IFN-γ, IL-1β, TGF-β1, IL-8, IL-12 and LIF. The amount of IL-6 present in the supernatants was measured using an IL-6 ELISA developed in our laboratory (Sukkar et al., 2000).

Immunohistochemistry

Immunohistochemistry was performed on HASM cells grown to confluence on glass coverslips in 10% FBS DMEM as described previously (Triantafilou et al., 2001).

Biotinylation and Immunoprecipitation

HASM cells growing in culture were washed on ice with PBS, then surface biotinylated by incubation with 0.5 mg/mL Sulfo-N-hydroxysuccinimide-biotin in PBS on ice for 15 min. Cells were washed, then lysed in 10 mM Tris pH 7.4, 0.14 M NaCl, 3 mM MgCl2, 1 mM PMSF and 0.5% Triton X-100. The lysate was briefly incubated with Protein G agarose, then centrifuged to remove agarose, nuclei and cell debris. The supernatant was incubated with the OX40L-specific monoclonal antibody 5A8 overnight (4° C.), then with Protein G agarose for 3 h. The agarose beads were then extensively washed according to the manufacturer's instructions, and extracted with electrophoresis buffer. Samples were boiled for 5 min, loaded onto a 15% polyacrylamide gel and separated by electrophoresis. Gels were electro-blotted to nitrocellulose membranes, membranes were blocked overnight with 1% FBS, then washed and incubated with Streptavidin HRP conjugate for 1 h. Biotinylated proteins were visualized using enhanced chemiluminescence. Two major protein bands were detected, one at the target MW of 30000.

Western Analysis

HASM cells were grown to confluence for 7 days in 10% FCS DMEM, then quiesced for 24 h in 1% FCS DMEM. OX40:Fc, or the Fc fragment control, were added to the cells at 1 μg/ml for durations from 5 min to 24 h. At completion of these incubations, cells were washed in ice-cold PBS and kept on ice for extraction. Cells were lyzed in extraction buffer (30 mM Tris pH 7.4, 1 mM EDTA, 2 mM benzamidine, 0.5 mM PMSF) by scraping from the culture wells, multiple pipeting and incubation for 30 min on ice. Cell debris was removed by centrifugation (5060×g, 5 min, 4° C.), then the supernatants were fractionated by centrifugation (14000×g, 60 min, 4° C.). For membrane fractions, the supernatants were removed and the pellet dissolved in extraction buffer containing 0.5% Triton X-100. For Western analysis, cell extracts containing 10 μg total protein were separated on 10% polyacrylamide gels, transferred to nitrocellulose membranes and blocked overnight in 5% (w/v) skim milk solution. Primary and secondary antibodies were diluted according to the manufacturers' recommendations. The blots were visualized by enhanced chemiluminescence.

Laser Capture Microdissection

Laser capture microdissection was conducted using five μm serial sections of human airway tissue which were cut and stained with (a) mouse anti-human a smooth muscle actin and the Dako LSAB2 new fuschin detection system and counter-stained with Mayer's hematoxylin for the identification of the different cell types or (b) rapid Mayer's hematoxylin and eosin (H&E), and then viewed through a visualiser on the LCM microscope (Roadmap image). For capturing, the H&E stained section was viewed without the visualiser showing the (c) before image, (d) after image (where the captured cells are removed) and (e) cap image of the captured cells.

Statistics

Analysis of variance (ANOVA) using repeated measures and the Fisher protected least squares difference post test was performed on the results for real time RT-PCR, ELISA and Western blots. In all cases a P value of less than 0.05 was considered significant.

### Example 1

### Identification of the OX40L mRNA in Human Airway Smooth Muscle Cells

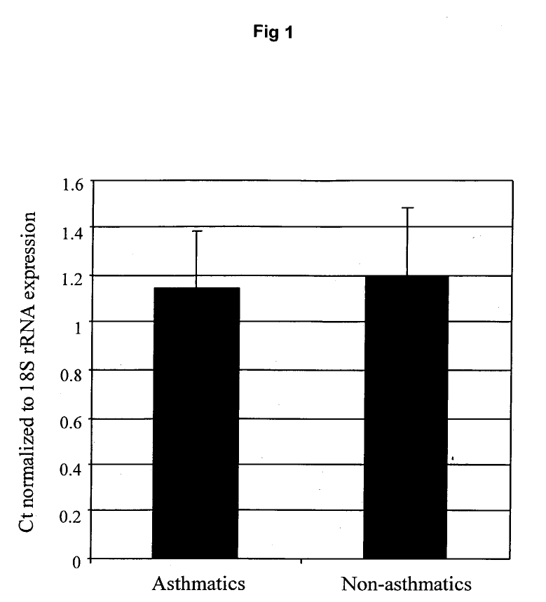

To search for expression of the OX40L gene in HASM cells, Northern analysis was performed. Total RNA from three patients was isolated from untreated cells, cells treated with atopic serum for 2 h or 24 h and cells treated with nonatopic serum for 2 h or 24 h. A probe specific for OX40L was used to detect the mRNA via hybridisation. The expression of OX40L mRNA was found to be below the detection limit of a Northern suggesting that this was a lowly expressed gene in these cells. Based on an understanding of the expression level of OX40L mRNA in these cells, real time RT-PCR was used to examine the expression of OX40L mRNA in non-asthmatic and asthmatic ASM cells. Expression of OX40L was normalized to the level of 18S rRNA in each reaction. OX40L mRNA was expressed in both non-asthmatic and asthmatic cells with equal abundance as indicated in FIG. 1. This confirmed that human ASM cells were capable of expressing OX40L mRNA and that the steady-state level of expression was low in comparison to rRNA.

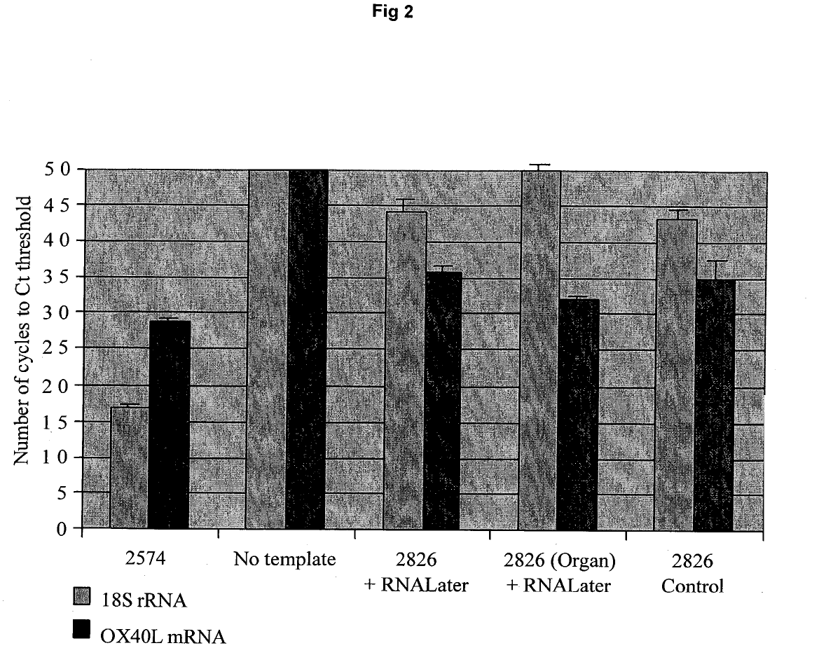

To determine whether the expression of the OX40L mRNA was a cell culture phenomenon, HASM cells were laser-captured from human biopsy samples. Total RNA was isolated and RT-PCR used to assess the expression profile of OX40L (FIG. 2B). FIG. 2 shows that the OX40L mRNA is expressed in cells present in the original patient biopsy and confirms that the observed expression in cultured cells is representative of OX40L expression level in the tissue source.

### Example 2

### Immunodetection of OX40L Surface Protein on Cultured Human ASM Cells

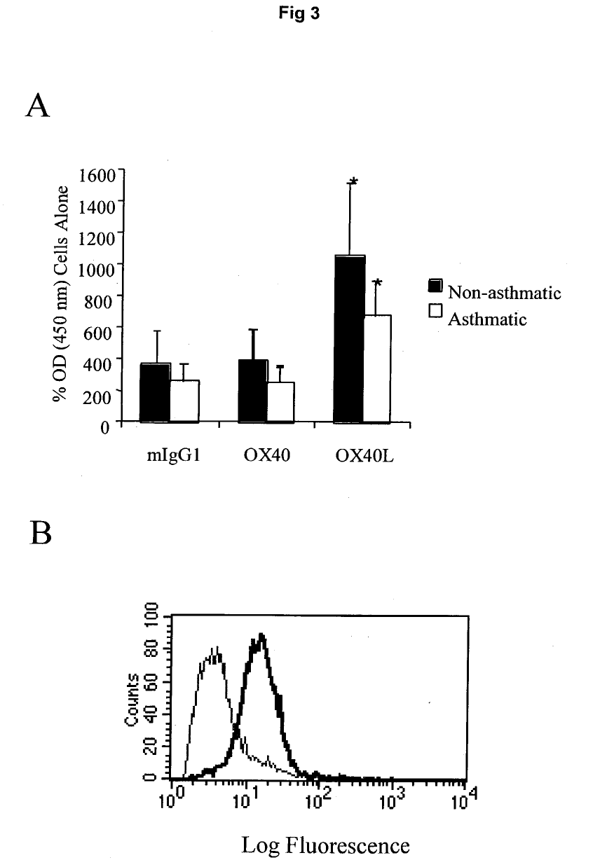

The expression of the OX40L protein on the surface of the HASM cells in culture was measured using a modified ELISA. Both non-asthmatic and asthmatic ASM cells express OX40L, measured with 5A8, on the cell surface (FIG. 3). The results are expressed as a percentage of absorbance (450 nm) of cells alone, which was set at 100%. The absorbance of OX40L was significantly greater than the IgG₁ isotype control for both cell types (P<0.05). Surface expression of OX40 was not detected in this assay on either cell type. Positive and negative controls of antibodies to detect MHC I and II, respectively, were used for the ELISA assay, confirming the specificity of this detection system. In addition, the same OX40L-specific antibody was used to immunoprecipitate a surface protein of 30 kDa from HASM cells.

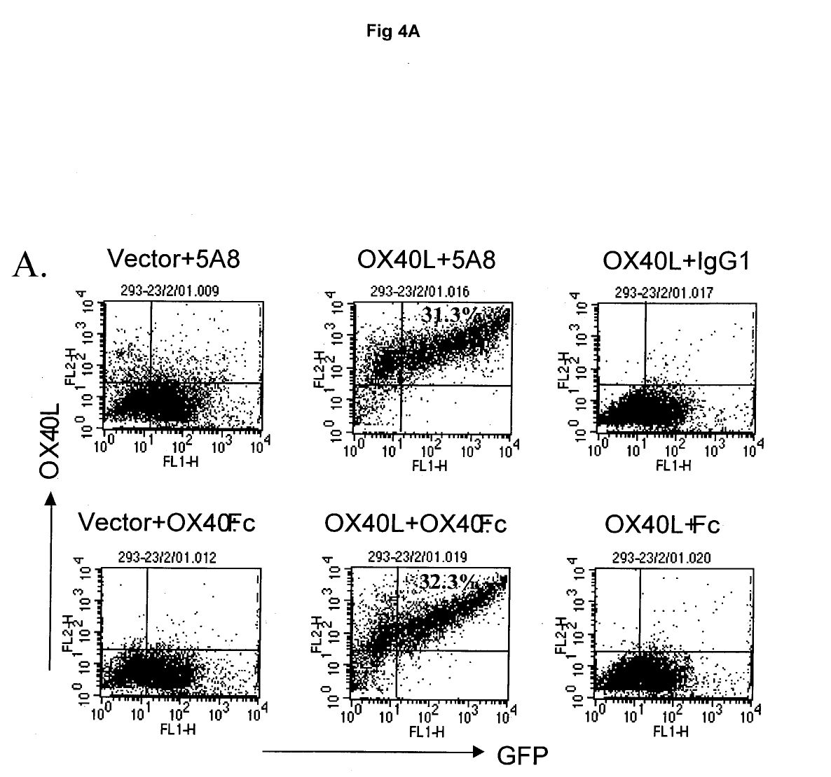

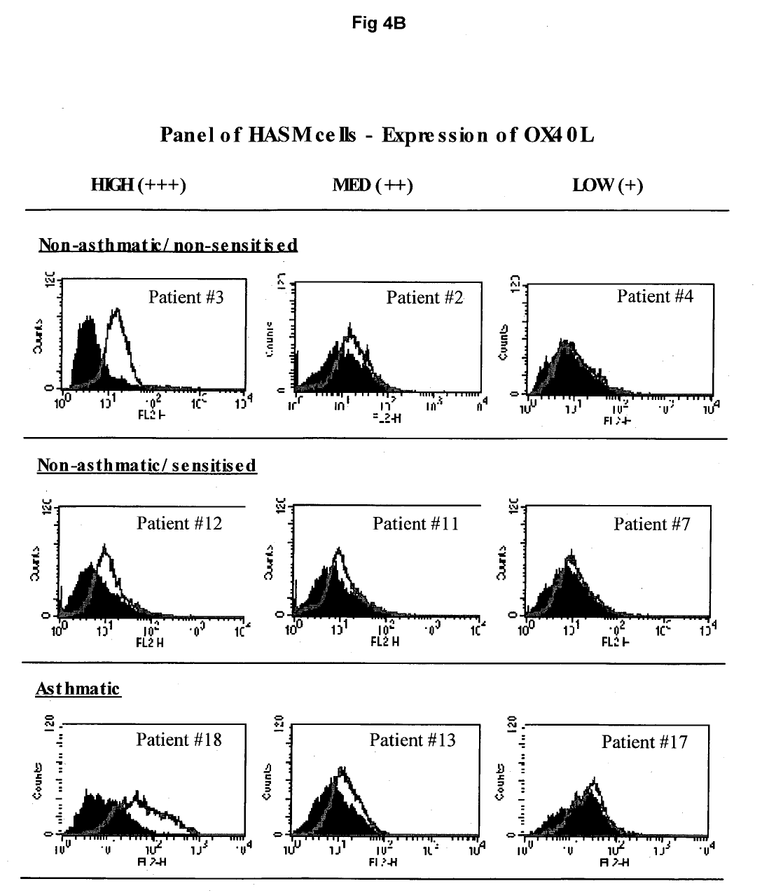

In a second detection assay, surface expression of OX40L on HASMs was measured by flow cytometry using 5A8 and the recombinant human OX40:Fc construct (FIG. 4A). The OX40:Fc is a recombinant fusion protein of human OX40, specifically amino acids 1-220, and the Fe portion of human IgG. The utility of the above detection assays was initially confirmed using each to identify OX40L expressed on human embryonic kidney cells transfected with an OX40L expression plasmid and HUVECs, the latter of which have been shown to express high levels of the OX40L. Surface expression was then detected on asthmatic, non-asthmatic/non-sensitised and asthmatic ASM cells using the OX40:Fc detection system (FIG. 4B). Human IgG Fc fragment did not bind to the surface of the HASM cells indicating that the OX40:Fc binding was specific for the OX40-OX40L interaction. A panel of non-asthmatic and asthmatic cells were further assessed for surface expression of OX40L, OX40, CD40, CD40L, MHC I and MHC II. The surface expression was classified according to the percentage of expression relative to the isotype control cells gated at 50% expressed as −(<55%); +(55-70%); ++(71-85%) or +++(>85%) (Table 1). As expected, all of the HASM cells expressed the MIHC I surface protein but did not display expression of MHC II or CD40L. Consistent with earlier reports the HASM cells expressed the TNF receptor family member CD40. Examination of the OX40-OX40L pair indicated that all HASM cells expressed OX40L, however no surface expression of OX40 was detected. As with the mRNA analysis, there was no observed difference in the surface expression pattern of OX40L between the classes of patient samples.

TABLE 1
Characterisation of HASM cell surface molecules.
Analysis of cell surface molecules present on non-asthmatic and asthmatic ASM cells.
Presence of the molecules was classified according to the percentage of fluorescence
relative to the isotype control cells gated at 50% expressed as −(<55%); +(55-70%);
++(71-85%) or +++(>85%).

| | OX40L | OX40 | CD40 | CD40L | MCHI | MCHII |
|---|---|---|---|---|---|---|
| Non-Asthmatic/Non-Sensitised | | | | | | |
| 1. (passage #7) Unknown sex & age, Transplant Donor | ++ | − | ++ | − | +++ | − |
| 2. (P6) M, 38, Cystic Fibrosis (CF), Transplant | ++ | − | + | − | +++ | − |
| 3. (P8) F, 52, Emphysema, Transplant | +++ | − | ++ | NT | +++ | ++ |
| 4. (P9) M, 61, Emphysema, Transplant | + | − | ++ | − | +++ | − |
| 5. (P5) M, 60, Emphysema, Transplant | +++ | − | ++ | − | +++ | − |
| 6. (P5) M, 40, Brochiectasis, Transplant | ++ | − | ++ | − | +++ | − |
| Non-Asthmatic/Sensitised | | | | | | |
| 7. (P8) M, 50, ^{α}-1 anti-trypsin deficiency, Transplant, (HDM) | + | − | + | − | +++ | − |
| 8. (P5) F, 55, Emphysema, Transplant, (Cat Hair) | + | − | + | NT | +++ | − |
| 9. (P4) F, 39, CF, Transplant, (HDM, Alternaria, Aspergillus) | + | − | + | − | +++ | − |
| 10. (P4) F, 73, Carcinoma, Resection, (HDM) | + | − | +− | +++ | − | |
| 11. (P4) F, 62, Cancer, Resection | + | − | + | − | +++ | − |
| 12. (P5) M, 53, Emphysema, Transplant, (HDM) | +++ | − | + | − | +++ | − |
| Asthmatic | | | | | | |
| 13. (P6) M, 33, Transplant, (HDM, Cat Hair, Alternaria, Timothy Grass) | ++ | − | ++ | − | +++ | − |
| 14. (P6) M, 15, Transplant Donor | ++ | − | + | − | +++ | − |
| 15. (P7) M, 44, Transplant, Emphysema, Ex-smoker (HDM) | ++ | − | ++ | − | +++ | − |
| 16. (P8) M, 25, Biopsy | ++ | − | + | − | +++ | − |
| 17. (P6) M, 56, Biopsy | + | − | + | − | +++ | − |
| 18. (P6) M, 30, Biopsy, Ex-smoker | +++ | − | ++ | − | +++ | − |

Notes:
Sensitisation status to five common allergens: House Dust Mite (HDM); Cat Hair; Timothy Grass; and the moulds Alternaria and Aspergillus.
NT; not tested.

### Example 3

### OX40 and OX40L Immunohistochemistry on Cultured Human Airway Smooth Muscle Cells

Immunohistochemistry using OX40:Fc, followed by an anti-human Ig Fc fragment-PE conjugated secondary antibody, incubated with fixed ASM cells grown to confluence on glass coverslips demonstrated OX40L expression on non-asthmatic and asthmatic ASMs with no discernible difference in the degree of expression between the cell types. In this study photographs were taken that were representative of the results obtained using immunohistochemistry to detect surface expression of the OX40L on both asthmatic and non-asthmatic human airway smooth muscle cells grown on coverslips. The study included cells stained with anti-human Fc secondary antibody alone, with human IgG Fc fragment and anti-human Fc secondary antibody or with OC40:Fc and anti-human Fc secondary antibody. The staining was representative of results seen with 4 patients. The cells stained with anti-human Fc secondary antibody alone and human IgG Fc fragment and anti-human Fc Secondary antibody displayed background levels of signal. In contrast, cells stained with OX40:Fc and anti-human Fc secondary antibody showed bright signals on normal and asthmatic airway smooth muscle cells indicating expression of the OX40L on these cells. This confirmed OX40L expression on adherent non-sensitised non-asthmatic, sensitised non-asthmatic and asthmatic cells grown in both nonatopic and atopic serum.

Immunohistochemistry on HASM cells grown to confluence on glass coverslips in 10% FBS DMEM indicated that asthmatic cells, but not non-asthmatic cells, secrete OX40 receptor into the matrix surrounding the cells. OX40 was detected using the commercial monoclonal antibody clone ACT35 and an anti-mouse-FITC-conjugated secondary antibody or a PE-conjugated form of this antibody. The cells were ethanol fixed before staining was commenced. The expression of OX40 was not cell-associated and remained in the matrix even after the cells were washed from the surface of the coverslip. These results indicate that the ASM cell in culture derived from asthmatic tissue samples have the capacity to produce and secrete (or shed) the OX40 receptor.

### Example 4

### Cell Signalling in Human Airway Smooth Muscle Cells Treated with Soluble OX40:Fc

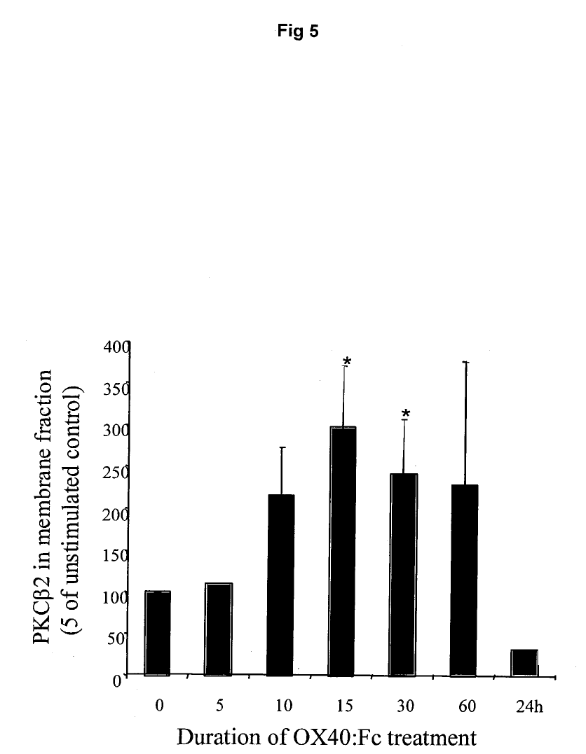

HASM cells were tested for their response to the addition of OX40 receptor, with the aim of testing whether OX40L has a signalling role. A range of signal transduction molecules were tested for response to incubation of cells with OX40 receptor. An initial screen of six PKC isoforms and MAP kinase showed that PKCβ2 was translocated to the membrane in response to OX40. This result was confirmed in two other patients. A Western blot was performed to detect the level of PKCβ2 in the membrane fraction of OX40L-expressing human airway smooth muscle cells. This Western blot provided the level of PKCβ2 protein at 0 minutes, 5 minutes, 15 minutes, 30 minutes, 60 minutes and 24 hours after addition of OX40:Fc. The level of PKCβ2 increased from 0-15 minutes and then progressively declined until 24 hours. The lowest level of PKCβ2 was detected at 24 hours. PKCβ2 was maximally activated at 15 min (3-fold increase in membrane fraction) (FIG. 5), while other PKC isoforms, namely PKCα and PKCβ1, were not activated. There was no response to incubation with the Fe fragment control. This indicated that PKCβ2 was consistently and rapidly translocated to the cell membrane following engagement of OX40L. There are no previous reports of PKCβ2 up-regulation in response to OX40L. The β1 and β2 isoforms of PKC are expressed through alternative splicing of the PKCβ gene. PKCβ2 is one of the isoforms of PKC that are activated by calcium and diacylglycerol. PKCβ2 has been most clearly associated with activation of cells by insulin (Arnold et al., 1993; Chalfant et al., 1998) and high glucose levels (Pirags et al., 1996). Ishii and colleagues (1996) showed that the harmful effects of diabetes in rats could be ameliorated by administration of a specific PKCβ inhibitor. PKCβ2 is also implicated in the control of structural proteins. It is associated with the microtubule cytoskeleton in resting cells (Kiley et al., 1995), and with the actin cytoskeleton (Faux and Scott, 1996). PKCβ2 also acts on structural proteins in its role in cell cycle progression (Gokmenpolar et al., 1998). These latter functions may be an important feature that enables OX40 stimulation to enhance the cell cycle turnover of cells.

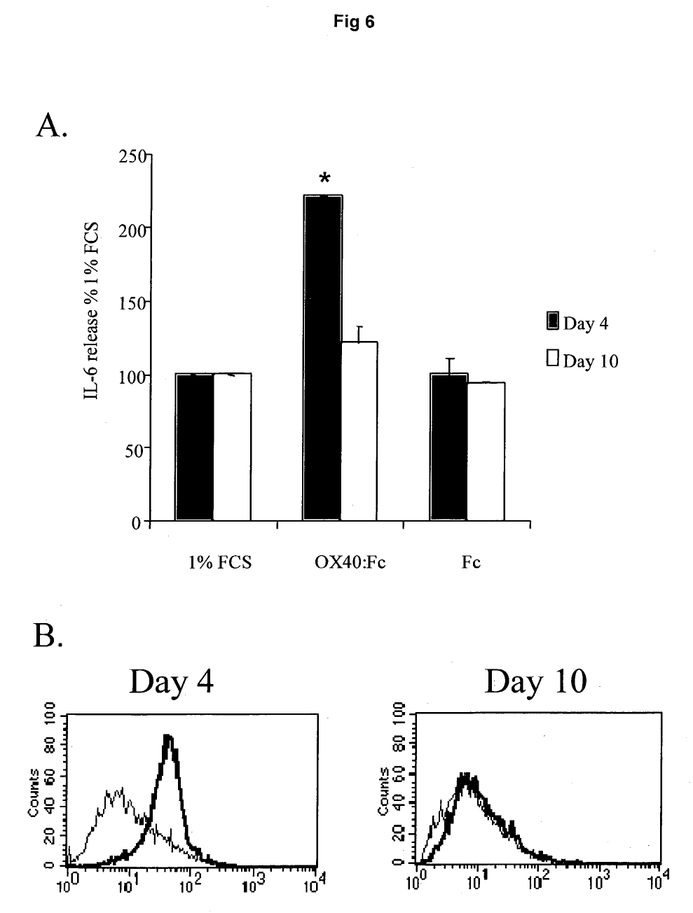

The supernatants collected from HASM cells grown in six-well plates treated with OX40:Fc or human Fe fragment for 24 h were assayed for cytokine levels using ELISA kits. Of the panel of cytokines tested only IL-6 release was altered by incubation of the cells with OX40:Fc. In all patients examined incubation of the ASM cells with OX40:Fc caused a significant increase in the release of IL-6 from the cells. In contrast, incubating the cells with human IgG Fe fragment did not increase the amount of IL-6 released (FIG. 6A). When cells from the same patient were treated with OX40:Fc in a sub-confluent or confluent state, a greater release of IL-6 was observed from the sub-confluent cells. The level of surface expression of OX40L was measured by flow cytometry on both cell populations (FIG. 6B). When the level of surface expression detected was high (day 4), the amount of IL-6 released into the supernatant was also high. On day 10, the level of surface expression had decreased and the amount of IL-6 released, although still greater than control, was less than observed on day 4. This indicates that the production of IL-6 by the ASM cells, upon engagement of the OX40L by the OX40 receptor, correlates with the surface expression of OX40L. Lazaar and colleagues reported a similar outcome when ASM were stimulated with recombinant CD40L (Lazaar et al., 1998). IL-6 has a number of proinflammatory properties likely to be important in airway inflammation during asthma, including the differentiation of B cells into antibody-producing cells (Muraguchi et al., 1988), up-regulation of IL-4-dependent immunoglobulin E production (Sanchez-Guerrero et al., 1997) and stimulation of T cell proliferation (Uyttenhove et al., 1988). In some cell types, IL-6 has been reported to have anti-apoptotic properties (Irvin et al., 2001; Kuo et al., 2001) and, while not wishing to be bound by theory, this may be the mechanism by which OX40L averts apoptosis in the stimulated cells.

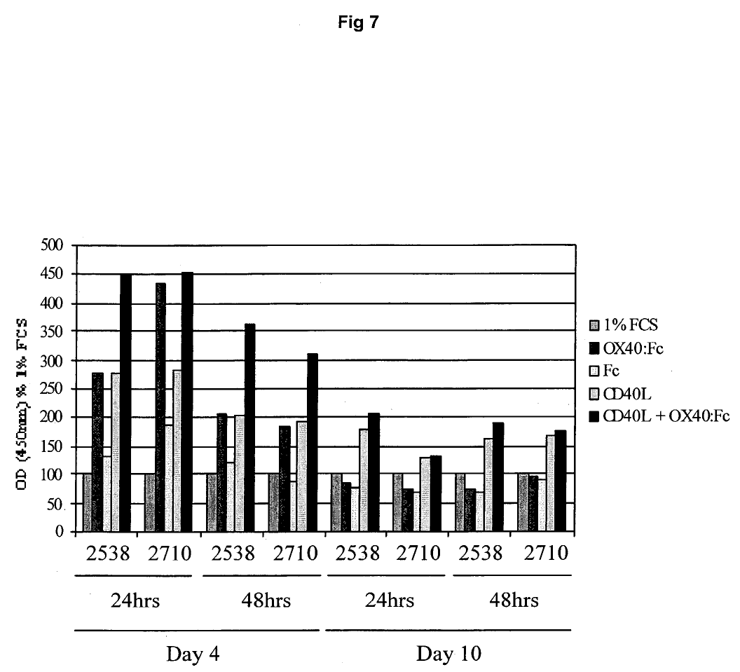

Based on the above observations, the response of ASM cells to stimulation with OX40:Fc and recombinant CD40L alone and in combination was examined. HASM cells were seeded at 1×10⁴ cells/cm² in 96 well plates and grown to sub-confluence (Day 4) or confluence (Day 10). The cells were then quiesced for 24 h with 1% FCS after which the OX40:Fc construct, an Fe control construct, recombinant human soluble CD40L (CD40L), and OX40:Fc in combination with CD40L were added to the cells at saturating concentrations (1 ug/ml). TNF-α alone (10 ng/ml) and in combination with OX40:Fc were also added to the quiesced cells. Supernatants were collected 24 and 48 h after addition of the constructs/cytokines to assay expression of IL-6 by ELISA (FIG. 7). As expected, treatment of ASM cells with TNF-α alone resulted in a substantial increase in the release of IL-6. Stimulation of these cells with either OX40:Fc or CD40L resulted in a two to three fold increase in IL-6 production, while treatment with both molecules in combination produced an additive increase in soluble IL-6. These data indicate that co-stimulation of the ASM cells through the OX40L and CD40 receptor results in the release of an amount of IL-6 equivalent to the sum of that released from ASM cells treated independently with either OX40:Fc or CD40L. This additive stimulation of IL-6 release was more pronounced in ASM cells displaying a higher level of surface expression of OX40L.

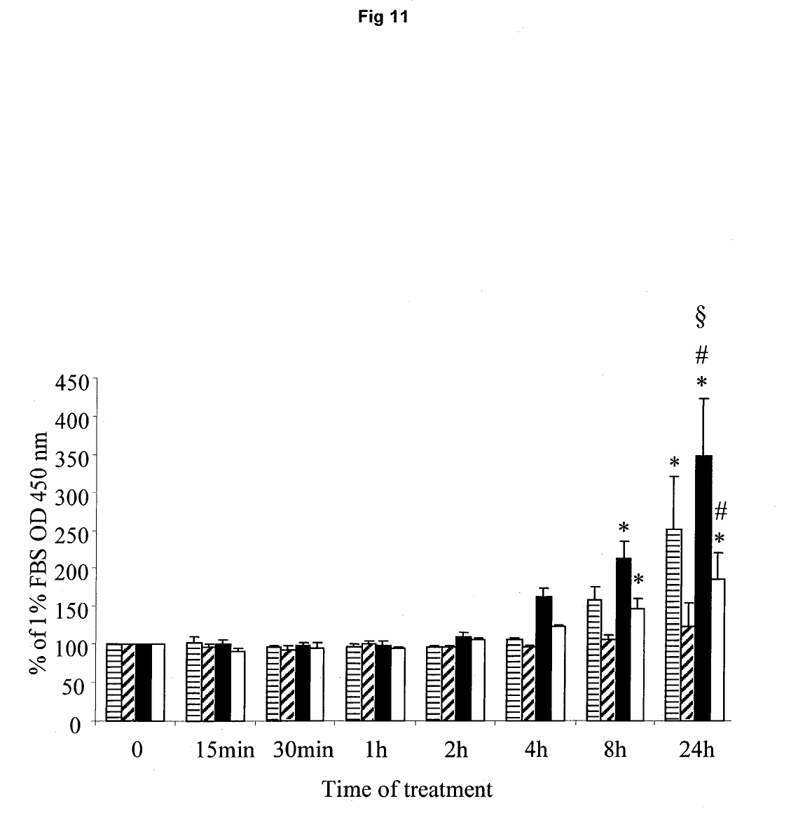

The time course of IL-6 release over a 24 h period, in response to OX40L stimulation, from asthmatic and non-asthmatic ASM cells was examined to determine if the dynamics of the IL-6 release altered between the cell types. An increase in IL-6 release was detected after 4 h in both the asthmatic and the non-asthmatic cells (FIG. 11). Levels significantly greater than the amount of IL-6 released at time 0 were seen at 8 and 24 h in response to OX40:Fc treatment in both the asthmatic and the non-asthmatic cells (P<0.01). In both cell types the response to OX40:Fc was greater than the response seen with the control Fe fragment (P<0.01). In the asthmatics a significant increase in IL-6 release at 24 h, compared to time 0, was seen with the Fe fragment alone (P<0.01). The overall release of IL-6 from the asthmatic cells was significantly greater than the non-asthmatic cells (P<0.02). Therefore the asthmatic ASM cells produced significantly greater amounts of IL-6 following OX40L stimulation than the non-asthmatic cells. These results suggest that the asthmatic cells have an enhanced response to the signaling events following OX40L stimulation. The slight response to the Fe control in the asthmatic cells suggests these cells also respond to other stimuli to which the non-asthmatic cells are non-responsive.

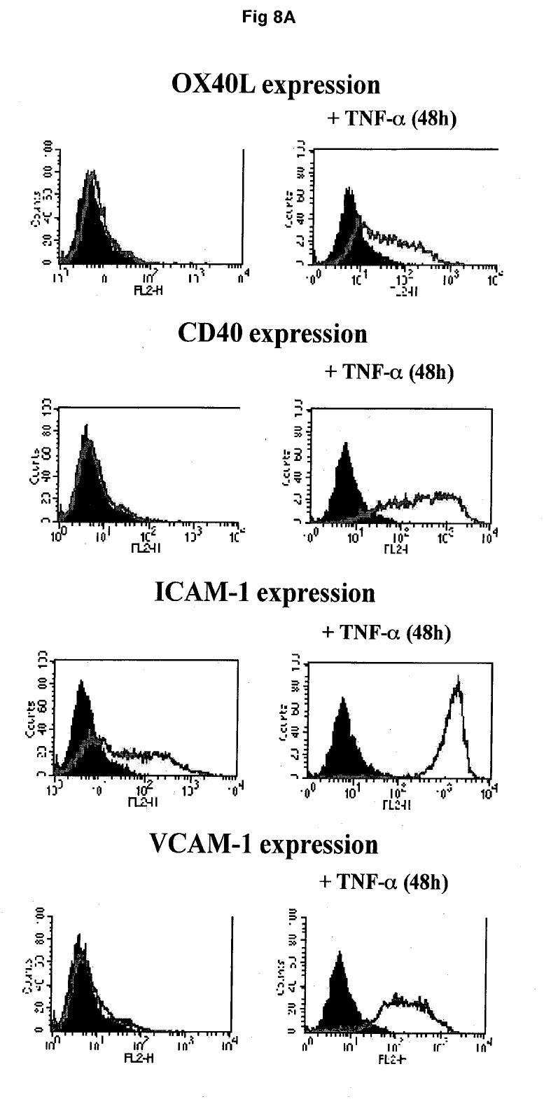

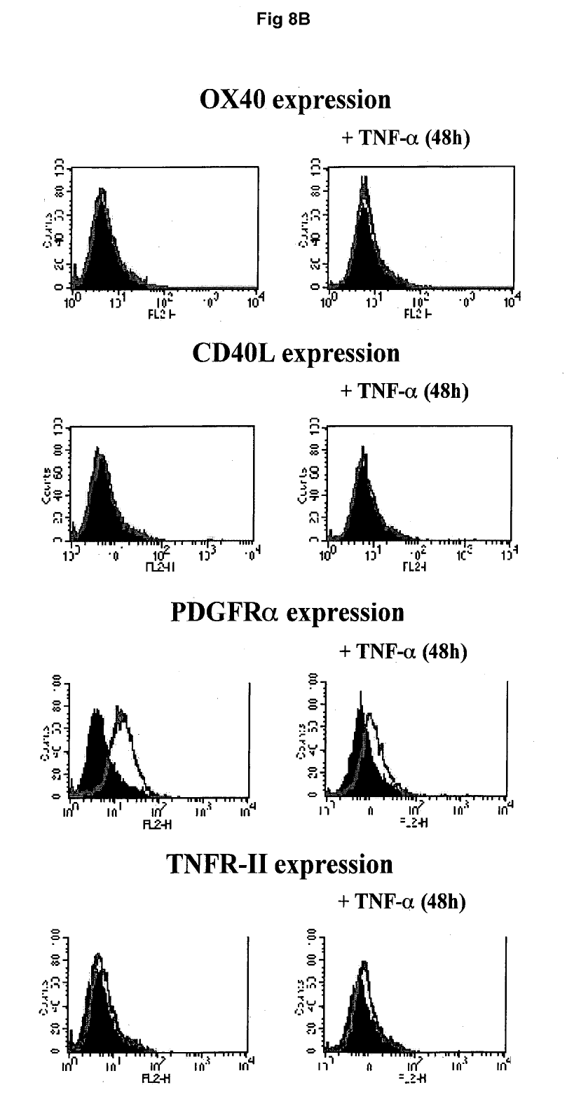

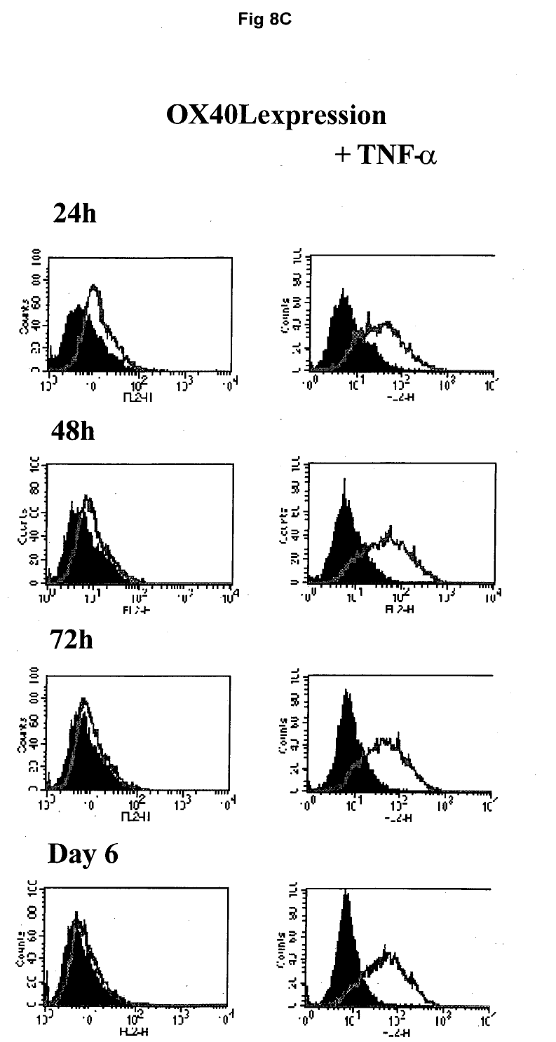

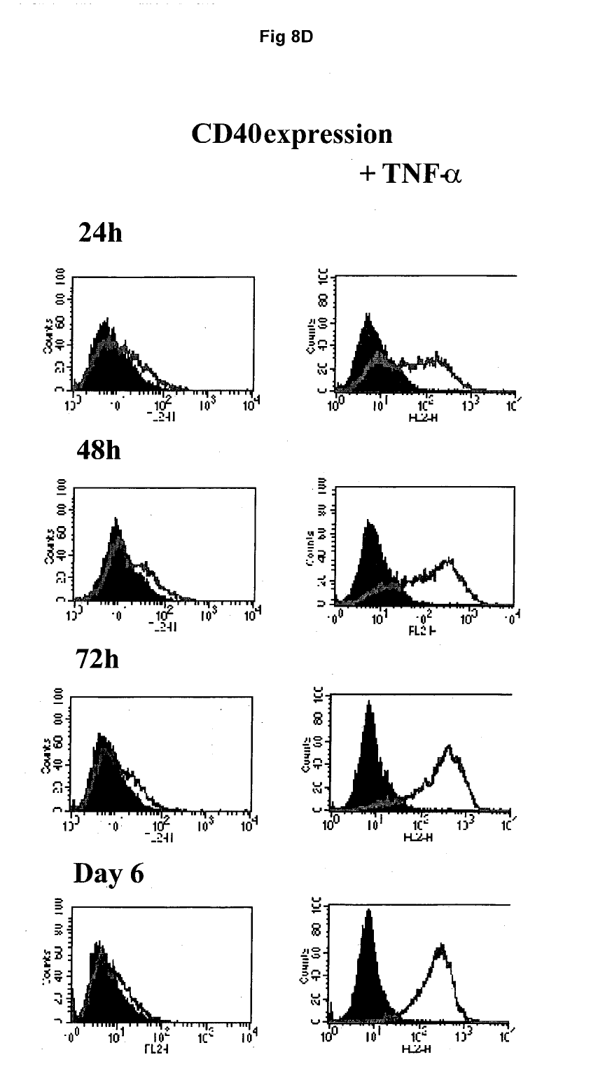

To further examine the effect of the pro-inflammatory cytokine TNFα on the CD40-OX40L inflammatory cascade in HASM cells, these cells were treated with TNFα and surface marker expression analysed. FIGS. 8A and B indicate that HASM cells treated with TNFα for 48 h show up-regulation of OX40L and CD40 but not TNF RII, OX40 or CD40L. In addition, these cells respond to TNFα by activating expression of adhesion molecules ICAM-1 and VCAM-I at their cell surface. A time course analysis of the response of HASM cells to TNFα showed that induction of OX40L and CD40 occurs within 24 h and that the increased levels of these TNF family members is maintained out to 6 days (FIGS. 8C and D). These observations indicate that the HASM cell maintains the ability to express cell surface markers consistent with cell adhesion and an inflammatory role.

### Example 5

### Phenotypic Modulation of ASM Cells in Response to Treatment with OX40:Fc

The sequential expression of CD40-CD40L and OX40-OX40L during T cell-B cell/APC interactions has been well characterized (Gramaglia et al., 1998; Walker et al., 1999; Murata et al., 2000) and some researchers suggest that the interaction of OX40 with its ligand is involved in T cell priming rather than the initial stimulatory signal (Chen et al., 1999; Lane, 2000; Murata et al., 2000). OX40L expression on cells has been reported at the sites of several inflammatory diseases, including APCs from the nervous system and brain microglia/macrophages of mice with actively induced EAE (Weinberg et al., 1999), synovial tissue from patients with rheumatoid arthritis (Yoshioka et al., 2000) and vascular endothelial cells from patients with systemic lupus erythematosus, eczema, erythema nodosum, muscular dystrophy and polymyositis (Matsumura et al., 1997). Although the role of OX40L in these disease processes has not been elucidated it has been suggested that OX40L provides a signal that promotes proliferation, cytokine production and secretion of high levels of Ig (Baum et al., 1994; Stuber et al., 1995). The OX40L on the vascular endothelial cells is thought to interact with OX40 on T cells to assist in T cell migration from the blood stream into sites of inflammation (Matsumura et al., 1997). These potential roles for OX40L could all be involved in the pathogenesis of asthma.

A murine model of asthma OX40-deficient mice primed with ovalbumin was recently shown to have a reduced ability to mount a Th2 response to aerosolized antigen (Jember et al., 2001). These mice were impaired in their ability to produce IL-4 and IL-5 and had reduced lung inflammation. The authors suggest that OX40 on T cells plays an important role in the development of allergic asthma. The present finding of the presence of OX40L on ASM cells provides a second, previously unknown mechanism, for T cell priming thereby supporting the importance of OX40 on T cells in the development of asthma. Although we found no significant difference in the expression of OX40L on ASM cells from non-asthmatic and asthmatic patients the potential difference lies in the interaction with the T cells. Non-asthmatic individuals do not suffer the extravasational episodes experienced by the asthmatic patients and therefore do not have the T cell infiltration into the airway wall. The presence of the T cells in the immediate locality of the ASM cells makes OX40L mediated priming of the T cells an important part of the inflammatory process. In addition, the presence of the OX40L on HASM cells, and the demonstration in this application that OX40:Fc can signal through this ligand to induce IL-6 production and mediate translocation of PKCβ2 to the cellular membrane in a manner dependent on the surface expression level of OX40L, suggests that the HASM cell can participate in T cell priming, maintaining the longevity of T cells and reverse signalling events that modify the HASM cell in asthma.

Stimulation of OX40 on antigen-activated CD4⁺ T cells recently has been shown to enable the cells to progress through additional cell cycles without the expression of apoptotic markers compared to unstimulated cells (Weatherhill et al., 2001). Furthermore, OX40L-expressing cells have been shown to be able to transfer the molecule to normal CD4⁺ T cells (Baba et al., 2001). The transferred OX40L stabilized in the T cell membrane and was able to stimulate latently HIV-I-infected T cells to produce HIV-1 p24 protein. Thus, the OX40L expressed on the ASM cells may provide a stimulus for the OX40 on T cells that would promote the longevity of the immune response at the site of lung inflammation. The ASM may also be able to transfer the OX40L to the T cells and facilitate further enhancement of the immune response through direct T cell-T cell interactions.

Human ASM cells are able to produce cytokines, undergo proliferation and migrate in response to various stimuli. OX40L on the surface of the ASMs provides such a stimulus, as in this study we have shown that engagement of this molecule by its receptor leads to increased IL-6 release but does not affect the proliferation of the cells (data not shown). These potential roles for OX40L could all be involved in the pathogenesis of asthma.

### Example 6

### Identification of a Blocking Antibody for the OX40-OX40L Interaction

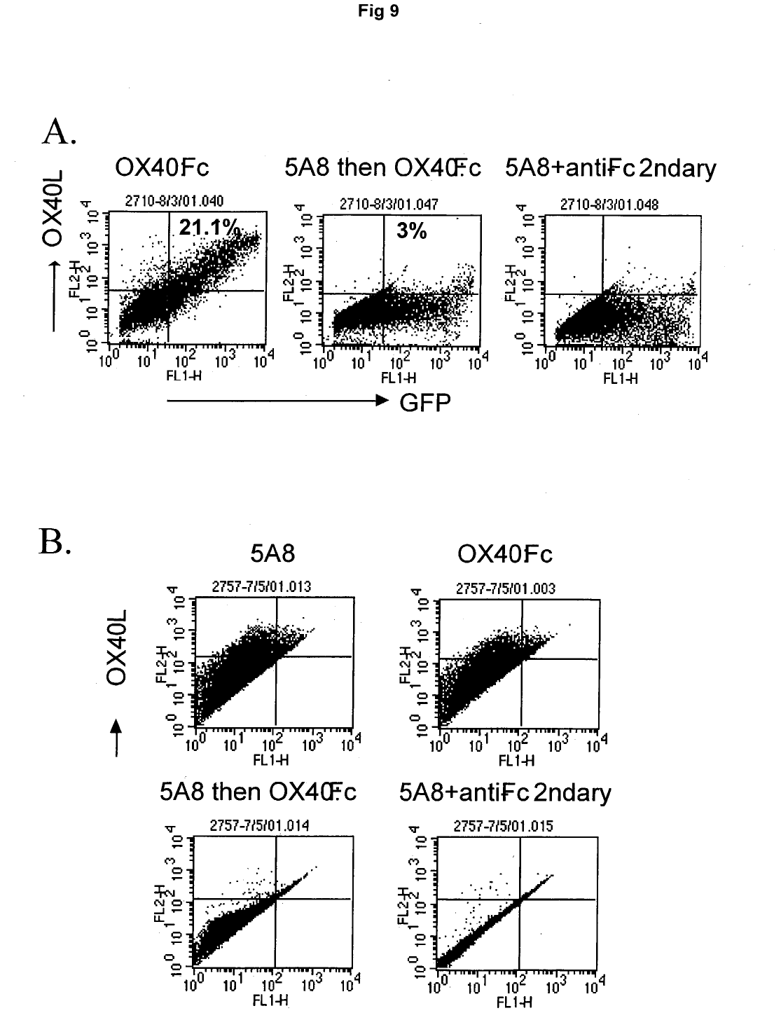

To identify a potential therapeutic for altering the engagement of the OX40L on the human ASM cells by OX40:Fc, 5A8 was tested as a blocking antibody. The ability of the 5A8 antibody to block the interaction of the OX40:Fc construct and OX40L was confirmed in both HASM cells over-expressing the OX40L from an expression plasmid (FIG. 9A) and in the high OX40L-expressing HASM cells (FIG. 9B). In this assay, HASM cells were harvested from flasks and the cell suspensions were incubated with high concentrations of 5A8 (10-100 μg/ml) for 10 min at room temperature prior to detection of OX40L expression using the OX40:Fc detection system described in Example 2. This result showed that molecules that can bind to OX40 can be used to interfere with the ligation of the OX40L on HASM cells by OX40 receptor and potentially block downstream signalling events mediated through this TNF ligand family member. Similarly, molecules that bind to OX40L can similarly be selected for their ability to block the binding of OX40 with OX40L.

### Example 7

### Expression of OX40L on Smooth Muscle Cells Derived from Human Colon

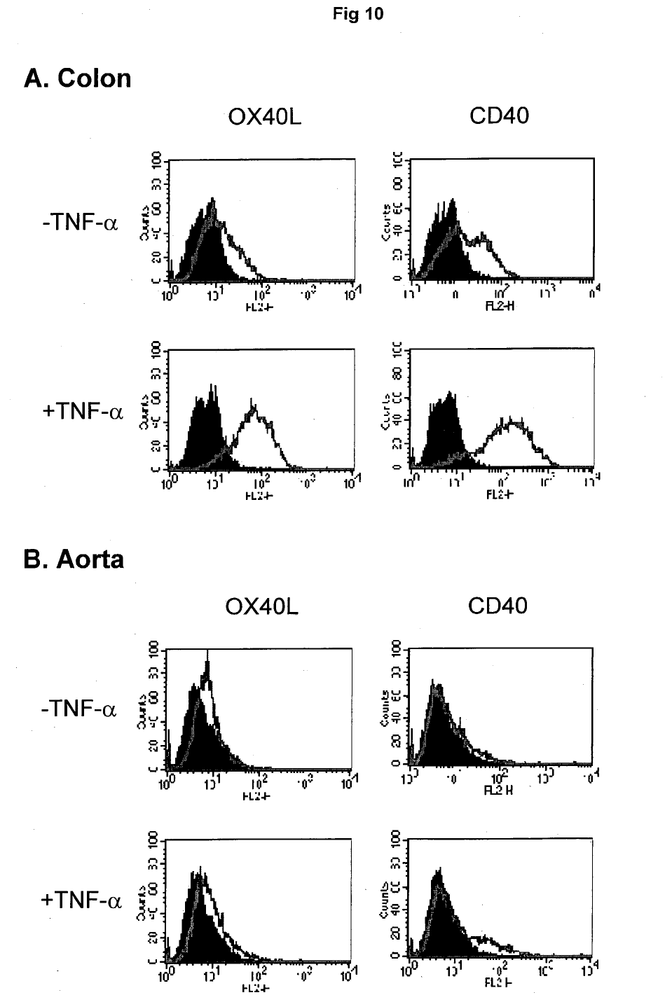

To determine whether the expression of OX40L, and its modulation by TNF-α, was specific to airway smooth muscles, the expression of OX40L was examined on primary human smooth muscle cells from other tissues. Colon smooth muscle cells from human patients were obtained commercially from Clonetics. Primary smooth muscle cells derived from the colon expressed OX40L on their cell surface with the level of OX40L being higher following treatment of these cells with TNF-α (FIG. 10A).

### Example 8

### Expression of OX40L on Smooth Muscle Cells Derived from Human Aorta

Primary smooth muscle cells from the aorta also expressed OX40L, but at lower levels than the colon smooth muscle cells (FIG. 10B). In addition, the expression of OX40L on the aortic smooth muscle cells was minimally upregulated by TNF-α. The results in examples 7 and 8 suggest that the OX40L protein is not only expressed on human airway smooth muscle cells but also smooth muscle cells derived from human colon and aorta.

### REFERENCES

Akiba, H. et al. CD28-independent costimulation of T cells by OX40 ligand and CD70 on activated B cells. Journal of Immunology 162, 7058-66 (1999).

Arnold, T. P. et al. Effects of insulin and phorbol esters on MARCKS (myristoylated alanine-rich C-kinase substrate) phosphorylation (and other parameters of protein kinase C activation) in rat adipocytes, rat soleus muscle and BC3H-1 myocytes. Biochemical Journal 295, 155-64 (1993).

Baba, E. et al. Functional CD4 T cells after intercellular molecular transfer of 0×40 ligand. Journal of Immunology 167, 875-83 (2001).

Baum, P. R. et al. Molecular characterization of murine and human OX40/OX40 ligand systems: identification of a human OX40 ligand as the HTLV-1-regulated protein gp34. EMBO Journal 13, 3992-4001 (1994).

Berberich, I., Shu, G. L. & Clark, E. A. Cross-linking CD40 on B cells rapidly activates nuclear factor-kappa B. Journal of Immunology 153, 4357-66 (1994).

Bissantz et al., J. Med. Chem. 43, 4759-4767 (2000).

Buenafe, A. C., Weinberg, A. D., Culbertson, N. E., Vandenbark, A. A. & Offner, H. V beta CDR3 motifs associated with BP recognition are enriched in OX-40+spinal cord T cells of Lewis rats with EAE. Journal of Neuroscience Research 44, 562-7 (1996).

Calderhead, D. M. et al. Cloning of mouse Ox40: a T cell activation marker that may mediate T-B cell interactions. Journal of Immunology 151, 5261-71 (1993).

Carlin, S., Yang, K. X., Donnelly, R. & Black, J. L. Protein kinase C isoforms in human airway smooth muscle cells: activation of PKC-zeta during proliferation. American Journal of Physiology 276, L506-12 (1999).

Chalfant, C. E. et al. Insulin Regulates Protein Kinase C-Beta-li Expression Through Enhanced Exon Inclusion in L6 Skeletal Muscle Cells—a Novel Mechanism of Insulin- and Insulin-Like Growth Factor-l-Induced 5′ Splice Site Selection. Journal of Biological Chemistry 273, 910-916 (1998).

Chen, A. I. et al. Ox40-ligand has a critical costimulatory role in dendritic cell:T cell interactions. Immunity 11, 689-98 (1999).

Durand-Arczynska, W., Marmy, N. & Durand, J. Caldesmon, calponin and alpha-smooth muscle actin expression in subcultured smooth muscle cells from human airways. Histochemistry 100, 465-471 (1993).

Evans, D. E., Munks, M. W., Purkerson, J. M. & Parker, D. C. Resting B lymphocytes as APC for naive T lymphocytes: dependence on CD40 ligand/CD40. Journal of Immunology 164, 688-97 (2000).

Faux, M. C. & Scott, J. D. More on target with protein phosphorylation—conferring specificity by location [Review]. Trends in Biochemical Sciences 21, 312-315 (1996).

Flynn, S., Toellner, K. M., Raykundalia, C., Goodall, M. & Lane, P. CD4 T cell cytokine differentiation: the B cell activation molecule, OX40 ligand, instructs CD4 T cells to express interleukin 4 and upregulates expression of the chemokine receptor, Blr-1. Journal of Experimental Medicine 188, 297-304 (1998).

Foy, T. M., Aruffo, A., Bajorath, J., Buhlmann, J. E. & Noelle, R. J. Immune regulation by CD40 and its ligand GP39. Annual Review of Immunology 14, 591-617 (1996).

Francis, D. A., Karras, J. G., Ke, X. Y., Sen, R. & Rothstein, T. L. Induction of the transcription factors NF-kappa B, AP-I and NF-AT during B cell stimulation through the CD40 receptor. International Immunology 7, 151-61 (1995).

Godfrey, W. R., Fagnoni, F. F., Harara, M. A., Buck, D. & Engleman, E. G. Identification of a human OX-40 ligand, a costimulator of CD4+ T cells with homology to tumor necrosis factor. Journal of Experimental Medicine 180, 757-62 (1994).

Gokmenpolar, Y. & Fields, A. P. Mapping of a Molecular Determinant For Protein Kinase C Beta(Ii) Isozyme Function. Journal of Biological Chemistry 273, 20261-20266 (1998).

Gramaglia, I., Weinberg, A. D., Lemon, M. & Croft, M. Ox-40 ligand: a potent costimulatory molecule for sustaining primary CD4 T cell responses. Journal of Immunology 161, 6510-7 (1998).

Grewal, I. S. & Flavell, R. A. CD40 and CD154 in cell-mediated immunity. Annual Review of Immunology 16, 111-35 (1998).

Hawker, K. M., Johnson, P. R., Hughes, J. M. & Black, J. L. Interleukin-4 inhibits mitogen-induced proliferation of human airway smooth muscle cells in culture. American Journal of Physiology 275, L469-77 (1998).

Hicks, C., Sleigh, M. & Chesterman, C. Quantitation of platelet derived growth factor receptors on cells from human arterial segments. Journal of Immunological Methods 170, 83-92 (1994).

Higgins, L. M. et al. Regulation of T cell activation in vitro and in vivo by targeting the OX40-OX40 ligand interaction: amelioration of ongoing inflammatory bowel disease with an OX40-IgG fusion protein, but not with an OX40 ligand-IgG fusion protein. Journal of Immunology 162, 486-93 (1999).

Houghten et al., Drug Discovery Today 5, 276-285 (2000).

Huo, L. & Rothstein, T. L. Receptor-specific induction of individual AP-1 components in B lymphocytes. Journal of Immunology 154, 3300-9 (1995).

Irvin, B. J., Hanson, C. L., Smith, L. H. & Daniels, C. K. Cyclic AMP- and IL6-signaling cross talk: comodulation of proliferation and apoptosis in the 7TD1 B cell hybridoma. Experimental Cell Research 265, 73-9 (2001).

Ishii, H. et al. Amelioration of Vascular Dysfunctions in Diabetic Rats By an Oral Pkc Beta Inhibitor. Science 272, 728-731 (1996).

Jember, A. G., Zuberi, R., Liu, F. T. & Croft, M. Development of allergic inflammation in a murine model of asthma is dependent on the costimulatory receptor OX40. Journal of Experimental Medicine 193, 387-92 (2001). Johnson, P. R., Armour, C. L., Carey, D. & Black, J. L. Heparin and PGE2 inhibit DNA synthesis in human airway smooth muscle cells in culture. American Journal of Physiology 269, L514-9 (1995).

Kiley, S. C. & Parker, P. J. Differential Localization of Protein Kinase C Isozymes in U937 Cells—Evidence For Distinct isozyme Functions During Monocyte Differentiation. Journal of Cell Science 108, 1003-1016 (1995).

Kirshenbaum et al., Curr. Opin. Struct. Biol 9, 530-535 (1999).

Kuo, M. L., Chuang, S. E., Lin, M. T. & Yang, S. Y. The involvement of PI 3-K/Akt-dependent up-regulation of Mcl-1 in the prevention of apoptosis of Hep3B cells by interleukin-6. Oncogene 20, 677-85 (2001).

Lalmanach-Girard, A. C., Chiles, T. C., Parker, D. C. & Rothstein, T. L. T cell-dependent induction of NF-kappa B in B cells. Journal of Experimental Medicine 177, 1215-9 (1993).

Lane, P. Role of OX40 signals in coordinating CD4 T cell selection, migration, and cytokine differentiation in T helper (Th)1 and Th2 cells. Journal of Experimental Medicine 191, 201-6 (2000).

Lazaar, A. L. et al. T lymphocytes adhere to airway smooth muscle cells via integrins and CD44 and induce smooth muscle cell DNA synthesis. Journal of Experimental Medicine 180, 807-16 (1994).

Lazaar, A. L., Reitz, H. E., Panettieri, R. A., Jr., Peters, S. P. & Pure, E. Antigen receptor-stimulated peripheral blood and bronchoalveolar lavage-derived T cells induce MHC class II and ICAM-1 expression on human airway smooth muscle. American Journal of Respiratory Cell & Molecular Biology 16, 38-45 (1997).

Lazaar, A. L. et al. CD40-mediated signal transduction in human airway smooth muscle. Journal of Immunology 161, 3120-7 (1998).

Matsumura, Y., Imura, A., Hori, T., Uchiyama, T. & Imamura, S. Localization of OX40/gp34 in inflammatory skin diseases: a clue to elucidate the interaction between activated T cells and endothelial cells in infiltration. Archives of Dermatological Research 289, 653-6 (1997).

Miura, S., et al. Molecular cloning and characterization of a novel glycoprotein, gp34, that is specifically induced by the human T-cell leukemia virus type I transactivator p40^{tax}. Molecular and Cellular Biology 11, 1313-25 (1991).

Muraguchi, A. et al. The essential role of B cell stimulatory factor 2 (BSF-2/IL-6) for the terminal differentiation of B cells. Journal of Experimental Medicine 167, 332-344 (1988).

Murata, K. et al. Impairment of antigen-presenting cell function in mice lacking expression of OX40 ligand. Journal of Experimental Medicine 191, 365-74 (2000).

Ohshima, Y. et al. Expression and function of OX40 ligand on human dendritic cells.

Journal of Immunology 159, 3838-48 (1997).

Pirags, V., Assert, R., Haupt, K., Schatz, H. & Pfeiffer, A. Activation of Human Platelet Protein Kinase C-Beta(2) in Vivo in Response to Acute Hyperglycemia. Experimental & Clinical Endocrinology & Diabetes 104, 431-440 (1996).

Sanchez-Guerrero, I. et al. Co-stimulation of cultured peripheral blood mononuclear cells from intrinsic asthmatics with exogenous recombinant IL-6 produce high levels of IL-4-dependent IgE. European Respiratory Journal 10, 2091-2096 (1997).

Stuber, E., Neurath, M., Calderhead, D., Fell, H. P. & Strober, W. Cross-linking of OX40 ligand, a member of the TNF/NGF cytokine family, induces proliferation and differentiation in murine splenic B cells. Immunity 2, 507-21 (1995).

Sukkar, M. B., Hughes, J. M., Johnson, P. R. & Armour, C. L. GM-CSF production from human airway smooth muscle cells is potentiated by human serum. Mediators of Inflammation 9, 161-8 (2000).

Tittle, T. V., Weinberg, A. D., Steinkeler, C. N. & Maziarz, R. T. Expression of the T-cell activation antigen, OX-40, identifies alloreactive T cells in acute graft-versus-host disease. Blood 89, 4652-8 (1997).

Toellner, K. M. et al. T helper 1 (Th1) and Th2 characteristics start to develop during T cell priming and are associated with an immediate ability to induce immunoglobulin class switching. Journal of Experimental Medicine 187, 1193-204 (1998).

Triantafilou, M., Wilson, K. M. & Triantafilou, K. Identification of Echovirus 1 and coxsackievirus A9 receptor molecules via a novel flow cytometric quantification method. Cytometry 43, 279-89 (2001).

Uyttenhove, C., Coulie, P. & Van Snick, J. T cell growth and differentiation induced by interleukin-HP1/IL-6, the murine hybridoma/plasmacytoma growth factor. Journal of Experimental Medicine 167, 1417-1427 (1988).

Van Gool, S. W., Vandenberghe, P., de Boer, M. & Ceuppens, J. L. CD80, CD86 and CD40 provide accessory signals in a multiple-step T-cell activation model. Immunological Reviews 153, 47-83 (1996).

Vetto, J. T. et al. Presence of the T-cell activation marker OX-40 on tumor infiltrating lymphocytes and draining lymph node cells from patients with melanoma and head and neck cancers. American Journal of Surgery 174, 258-65 (1997).

Walker, L. S. et al. Compromised OX40 function in CD28-deficient mice is linked with failure to develop CXC chemokine receptor 5-positive CD4 cells and germinal centers. Journal of Experimental Medicine 190, 1115-22 (1999).

Weatherill, A. R., Maxwell, J. R., Takahashi, C., Weinberg, A. D. & Vella, A. T. Ox40 ligation enhances cell cycle turnover of ag-activated cd4 t cells in vivo. Cellular Immunology 209, 63-75 (2001).

Weinberg, A. D. et al. OX-40 antibody enhances for autoantigen specific V beta 8.2+ T cells within the spinal cord of Lewis rats with autoimmune encephalomyelitis. Journal of Neuroscience Research 43, 42-9 (1996a).

Weinberg, A. D. et al. Selective depletion of myelin-reactive T cells with the anti-OX-40 antibody ameliorates autoimmune encephalomyelitis. Nature Medicine 2, 183-9 (1996b).

Weinberg, A. D., Wegmann, K. W., Funatake, C. & Whitham, R. H. Blocking OX-40/OX-40 ligand interaction in vitro and in vivo leads to decreased T cell function and amelioration of experimental allergic encephalomyelitis. Journal of Immunology 162, 1818-26 (1999).

Yoshioka, T. et al. Contribution of OX40/OX40 ligand interaction to the pathogenesis of rheumatoid arthritis. European Journal of Immunology 30, 2815-23 (2000).

What is claimed is:

1. A method of reducing inflammation in smooth muscle tissue in a subject, said method comprising administering to said subject an effective amount of an agent which inhibits OX40L binding to OX40.

2. A method of reducing the induction of IL6 production in smooth muscle tissue in a subject, said method comprising administering to said subject an effective amount of an agent which inhibits OX40L binding to OX40.

3. A method of inhibiting translocation of PKCβ2 to cellular membranes in smooth muscle cells comprising administering to said subject an effective amount of an agent which inhibits or blocks interaction between OX40 and OX40L.

4. A method of inhibiting T cell priming in inflammatory diseases or conditions of smooth muscle tissue in a subject, said method comprising administering to said subject an effective amount of an agent which inhibits or blocks interaction between OX40 and OX40L.

5. The method of any one of claims 1-4, wherein the smooth muscle cells are airway smooth muscle cells.

6. The method of any of claims 1-3, wherein the inflammatory disease or condition is asthma.

7. The method of any one of claims 1-4, wherein the agent is a monoclonal antibody, or fragment thereof, which specifically binds to OX40 or OX40L.

8. The method of any one of claims 1-7, wherein the agent is a recombinant antibody or recombinant antibody fragment which specifically binds to OX40 or OX40L.

9. The method of any one of claims 1-7, wherein the agent is a peptide or polypeptide fragment of OX40 which is capable of blocking the interaction of OX40 with OX40L, or is a mimetic of said peptide or polypeptide.

10. A method for inhibiting translocation of PKCβ2 to the cellular membranes of smooth muscle cells comprising OX40L, the method comprising the step of inhibiting the ability of OX40 to bind to OX40L.

11. The method of claim 10 wherein the smooth muscle cell is obtained from airway tissue, colon tissue or aorta tissue.

12. The method of claim 10 wherein the cellular membranes of a smooth muscle cell are part of intact smooth muscle cells.

13. The method of claim 12 wherein the muscle cells are in a tissue or tissue sample.

14. The method of claim 10 wherein the cellular membranes of a smooth muscle cell are part of a cell lysate.

15. The method of claim 10 wherein the inhibiting step comprises contacting the cell membranes with an agent capable of inhibiting OX40 binding to OX40L.

16. The method of claim 15 wherein the agent binds to OX40.

17. The method of claim 15 wherein the agent binds to OX40L.

18. The method of claim 15 wherein the agent is an antibody or antibody fragment capable of binding to OX40 or alternatively is an agent capable of binding to OX40L.

19. The method of claim 10 wherein the inhibiting step comprises inhibiting expression of OX40L and wherein the cellular membranes of smooth muscle cells comprise intact smooth muscle cells.

20. The method of claim 19 wherein the inhibiting step further comprises introducing a gene expression reduction agent into the cell,

21. The method of claim 20 wherein the wherein the gene expression reduction agent is selected from the group consisting of DNAzymes, ribozymes, antisense RNA, dominant negative polypeptides, genetic suppression elements or intracellular antibodies.

22. An antibody or antibody fragment capable of inhibiting OX40 binding to OX40L on smooth muscle cells.

23. A method of identifying an agent that is capable of inhibiting binding of OX40 and OX40L in a smooth muscle cell preparation, said method comprising the steps of:
(a) combing the agent with isolated smooth muscle cell membranes;
(b) adding OX40; and
(c) detecting binding of OX 40 to OX40L.

24. The method of claim 23 wherein a reduction in the binding of OX40 to OX40L identifies a candidate agent.

25. The method of claim 23 wherein either OX 40, OX40L or both are labelled to promote detection.

26. The method of claim 23 wherein the smooth muscle cell membranes are part of intact smooth muscle cells.

27. The method of claim 26 wherein the smooth muscle cells are part of a tissue sample.

28. A method of identifying a molecule capable of inhibiting the interaction between OX40 and OX40L in smooth muscle cells, said method comprising the steps of:
(a) introducing the molecule into a collection of smooth muscle cells obtained from a subject;
(b) treating the collection from (a) with labelled OX40;
(c) washing the treated collection from (b) to remove any unbound labelled OX40; and
(d) assessing the washed collection from (c) for a reduced level of labelled OX40 relative to a control level of labelled OX40.

29. A composition comprising OX40 protein or a fragment thereof and isolated smooth muscle cell membranes comprising OX40L.

30. The cells of claim 29 wherein the cells are obtained from airway passage tissue, colon tissue or aorta.

31. The composition of claim 29 wherein the isolated smooth muscle cell membranes are obtained from a cell lysate of smooth muscle cells.

32. The composition of claim 29 wherein the isolated smooth muscle cell membranes are part of intact isolated smooth muscle cells.

33. An OX40:Fc fusion protein of human OX40.

34. The OX40:Fc fusion protein of claim 33 wherein the fusion protein comprises at least a portion of the human OX40 sequence and the Fc portion of human IgG.

35. The fusion protein of claim 33 comprising amino acids I-220 of OX40.
